SEQUENCE LISTING (10 sequences)

SEQ ID NO: 1 - DNA, 34 bp, Artificial Sequence
  ChIP-Seq
agatcggaagagctcgtatgccgtcttctgcttg

SEQ ID NO: 2 - DNA, 17 bp, Artificial Sequence
  ChIP-Seq
tcgtatgccgtcttctg

SEQ ID NO: 3 - DNA, 17 bp, Artificial Sequence
  ChIP-Seq
ctgtaggcaccatcaat

SEQ ID NO: 4 - RNA, 22 nt, Artificial Sequence
  tsRNA EGFR/MET
ucccugguggucuagugguuag

SEQ ID NO: 5 - RNA, 22 nt, Artificial Sequence
  tsRNA BCL2
uaagccagggauuguggguucg

SEQ ID NO: 6 - RNA, 20 nt, Artificial Sequence
  tsRNA LINC00665
ggggguguagcucaguggua

SEQ ID NO: 7 - RNA, 87 nt, Artificial sequence
  Synthetic oligonucleotide
guguggugacccaguggccuaauggauaaggcaucagccuccggagcuggggauuguggguucgagucccaucugggucgcgugaga

SEQ ID NO: 8 - RNA, 85 nt, Artificial sequence
  Synthetic oligonucleotide
gucaacagcgccgcugguguagugguaucaugcaagauucccauucuugcgacccggguucgauucccgggcggcgcauaggcgu

SEQ ID NO: 9 - RNA, 76 nt, Artificial sequence
  Synthetic oligonucleotide
gggcguguggcguagucgguagcgcgcucccuuagugugggagagggcuccggaucgauuccugacucguccacca

SEQ ID NO: 10 - RNA, 22 nt, Artificial sequence
  Synthetic oligonucleotide
cgagucucgguggaaccuuugu

(19) United States
(12) Patent Application Publication
(10) Pub. No.: US 2022/0220476 A1
(43) Pub. Date: Jul. 14, 2022

(54) MEDIATORS OF GENE SILENCING

(71) Applicant: Cancer Research Technology Limited, London, Greater London (GB)

(72) Inventor: Monika Gullerova, Oxford, Oxfordshire (GB)

(21) Appl. No.: 17/609,691

(22) PCT Filed: May 22, 2020
(86) PCT No.: PCT/GB2020/051257

§ 371 Date: Nov. 8, 2021

(30) Foreign Application Priority Data
May 24, 2019 (GB) 1907380.8

(51) Int. Cl. C12N 15/113

(52) U.S. Cl. CPC C12N 15/113; C12N 2310/531; C12N 2310/14; C12N 2310/113

(57) ABSTRACT

The present invention relates to a method of inhibiting expression of a gene in a biological system. The method of the present invention comprises introducing a tRNA-derived polynucleotide into the biological system. The tRNA-derived polynucleotide of the present invention comprises a sequence that is complementary to an intronic region of the gene whose expression is to be inhibited.

### FIELD OF THE INVENTION

The present invention relates to methods of inhibiting expression of a gene in a biological system. The present invention further relates to tRNA-derived polynucleotides and their uses as a medicament.

### BACKGROUND TO THE INVENTION

Mammalian cells employ small RNAs (sRNAs) originating from various endogenous sources to regulate gene expression in a pathway known as RNA interference (RNAi). These sRNAs include microRNAs (miRNAs) and small interfering RNAs (siRNAs) which can regulate the expression of many protein coding genes post-transcriptionally in the cytoplasm.

The RNAi pathway is present in many eukaryotes and is initiated by the enzyme Dicer, which cleaves long double-stranded RNA (dsRNA) into shorter double-stranded fragments which are approximately 21 nucleotides in length. These short double-stranded fragments are known as siRNAs. Each siRNA is unwound to form two single-stranded RNAs, one of which (the guide strand) is incorporated into an RNA-induced silencing complex (RISC). The guide strand then pairs with complementary messenger RNA (mRNA) and cleavage is induced by Argonaute 2 (Ago2), the catalytic component of RISC, resulting in post-transcriptional gene silencing.

miRNAs are non-coding RNAs that are involved in the regulation of gene expression, particularly during development. RNAi includes the gene silencing effects of miRNAs as well as those triggered by dsRNAs, as discussed above. miRNAs, once processed by Drosha are bound and cleaved by Dicer to produce miRNAs which can be incorporated into RISC. However, unlike siRNA-loaded RISC, miRNA-loaded RISC scans mRNAs for potential complementary sequences and binds to the 3′ untranslated region of these mRNAs, thereby preventing translation.

Progress in the understanding surrounding the mechanisms underlying RNAi has been exploited in a number of fields including research, for example the study of gene function, and various therapeutic applications for the treatment of disease. Diseases linked to the activity of one or more genes should be well suited to RNAi-based therapies, since small RNAs directed towards these genes can be designed and used as therapeutic agents. These could include, for example, cancer, autoimmune diseases and neurodegenerative diseases such as Alzheimer's disease, for example. Clinical applications of RNAi have been used to treat age-related macular degeneration and further therapies are being developed in a range of therapeutic areas including the treatment of various viral infections, such as HIV. It would be advantageous to develop a method of inhibiting gene expression which could be used in the treatment of disease.

Transfer RNAs (tRNAs) are RNA molecules between around 75-90 nucleotides in length which act as adaptor molecules to carry amino acids to the codon of a mRNA. tRNAs typically adopt a cloverleaf structure which includes an acceptor stem, which binds an amino acid and an anticodon arm which is complementary to the mRNA codon. In addition to their role as adaptor molecules, tRNAs have recently been identified as a source of a novel class of regulatory RNA fragments with uncharacterised roles.

It is an object of the present invention to provide new molecules and methods of inhibiting gene expression.

### SUMMARY OF THE INVENTION

The present invention relates to new methods for mediating RNA interference, in particular gene silencing. RNAi is a biological process in which RNA molecules inhibit gene expression or translation, by neutralizing targeted mRNA molecules. Thus, methods for inhibiting expression of a gene are also provided. We also provide a novel species of isolated tRNA polynucleotides which mediate gene silencing and their use in methods for treating disease.

The invention is based, in part, on studies by the inventor in which she has shown that it is possible to reduce the mRNA levels of specific genes and subsequent protein levels following transfection of cells with tRNA-derived polynucleotides, particularly short fragments of tRNA that mediate gene silencing. These short fragments of tRNA are termed tsRNA (tRNA-derived sRNAs). tsRNA are different from tRNA halves and tRNA fragments which are both cleavage products of a tRNA clover leaf structure.

However, as explained herein, tsRNA are generated by Dicer dependent cleavage of an alternatively folded tRNA, namely a stem loop/hairpin structure. Unlike siRNA, tsRNA are generated in the nucleus by Dicer cleavage of hairpin-like tRNA. tsRNA target genes in nucleus. Thus, the invention does not relate to tRNA halves and tRNA fragments generated by cleavage of a tRNA clover leaf structure.

The inventor has shown that the endoribonuclease Dicer, a critical player in canonical RNAi, associates with actively transcribed tRNA genes, binds to tRNAs that are folded into a non-canonical secondary structure and processes them into tsRNAs. Furthermore, these Dicer-dependent tsRNAs are functional and specifically target the introns of many protein coding genes, leading to the degradation of their nascent RNA in an Argonaute 2 (Ago2)-dependent manner. This distinguishes the mechanism from other RNA mechanisms mediated by known siRNAs. Furthermore, tsRNAs act in the nucleus whereas other known siRNA act in the cytoplasm. Targeting of the introns of genes or long non-coding RNA leads to immediate degradation of the RNA as soon as it is made in the nucleus. The inventor has shown that tsRNA is generated not from mature tRNA, but from a distinct tRNA population which is folded into an alternative secondary structure, a structure that can be described as a stem loop/hairpin structure. These alternatively folded tRNAs are recognised by Dicer and processed into tsRNA in the nucleus. We thus propose a novel mechanism for Dicer-dependent regulation of gene expression that is mediated by tsRNAs through nascent RNA degradation and is distinct from well-known post-transcriptional or transcriptional gene silencing.

Importantly, the inventor has shown that tsRNA target genes are significantly associated with various diseases, such as cancer, which underpins the biological importance of this pathway. Thus, tsRNA as described herein can be used in the treatment of disease.

The inventor of the present invention has found that by introducing a tRNA-derived polynucleotide, e.g. a tsRNA, into a biological system, for example a cell, expression of a gene or of a long non-coding RNA can be inhibited, provided the tRNA-derived polynucleotide comprises a sequence that is partially or fully complementary to an intronic region of the gene. The inhibition of gene expression achieved by the present inventor using this method has been shown to be highly effective.

Without wishing to be bound by theory, it is the understanding of the inventor that the tsRNA regulates gene expression in the nucleus co-transcriptionally by targeting the introns of protein coding genes for nascent RNA degradation rather than requiring transcriptional repression and heterochromatin formation.

### DETAILED DESCRIPTION

The embodiments of the invention will now be further described. In the following passages, different embodiments are described. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012).

Polynucleotide

In a first aspect of the present invention there is provided an isolated tRNA-derived polynucleotide comprising a sequence that is complementary to an intronic region of a target gene or of a long non-coding RNA, in particular wherein said tRNA-derived polynucleotide is a tRNA fragment has 14 to 35 nucleotides (tsRNA).

The term “isolated” means that the tRNA-derived polynucleotide is isolated from its natural environment and not naturally occurring. This indicates the involvement of the hand of man. The term, when referring to nucleic acid molecules, means that the nucleic acid molecule or the polypeptide is at least substantially free from at least one other component with which they are naturally associated in nature and as found in nature.

As will be understood by the skilled person, the term “polynucleotide” refers to a linear polymer comprising covalently bonding nucleotide monomers. Polynucleotides include DNA and RNA, for example. In embodiments of the present invention, the polynucleotides are tRNA-derived.

Transfer RNAs (tRNAs) are RNA molecules between around 75-90 nucleotides in length which act as adaptor molecules to carry amino acids to the codon of an mRNA. In the present application, the term “tRNA-derived” is used to indicate that the polynucleotide may comprise all or part of the sequence of a naturally occurring tRNA or a modified sequence of all or a part of the sequence of a naturally occurring tRNA (for example all or part of the sequence of a tRNA which has one or more insertions, deletions or modifications). The tRNA derived polynucleotide may be chemically synthesized RNA or an analogue of a naturally occurring RNA. An analogue may differ from the RNA by the addition, deletion, substitution or alteration of one or more nucleotides.

The term “tRNA-derived polynucleotide” may also include artificial polynucleotides based on the sequence of a naturally occurring tRNA, for example or an artificial polynucleotide developed using bioinformatics algorithms based on features of naturally occurring tRNAs, for example the sequence of naturally occurring tRNAs.

In some embodiments of the present invention, the tRNA-derived polynucleotides may comprise all or part of the sequence of a naturally occurring tRNA or a modified sequence of all or part of the sequence of a naturally occurring tRNA (for example including one or more insertions, deletions or modifications of the naturally occurring sequence). Preferably, where a sequence includes a modification of the naturally occurring sequence of a tRNA, the modification is a conservative modification. The skilled person will appreciate that by utilising a conserved modification, the characteristics of the modified nucleotide are maintained or substantially maintained. The modified sequence may include one or more artificial, rather than naturally occurring, nucleotides, for example a locked nucleic acid (LNA).

In embodiments, the tRNA-derived polynucleotides comprise a sequence that is at least 50, 60, 70, 80, 90 or 95% complementary to a naturally occurring tRNA sequence. In embodiments, the tRNA-derived polynucleotides comprise a sequence that is at least 98 or 99% complementary to a naturally occurring tRNA sequence.

The present inventor has surprisingly found that the use of tRNA-derived sequences, especially those with high sequence similarity to naturally occurring tRNAs, results in effective inhibition of gene expression and expression of long non-coding RNAs.

In an embodiment of the present invention, the polynucleotide comprises tRNA. In such embodiments, the tRNA preferably comprises a stem-loop/hairpin structure. The present inventor has shown that in addition to forming the canonical cloverleaf structure, tRNAs can also form an alternative secondary structure, particularly a stem-loop structure. The present inventor has shown that it is this stem-loop structure which can be bound by the endoribonuclease Dicer, for subsequent processing and cleavage to yield tsRNA in the nucleus. Thus, the tsRNA described herein are produced by Dicer dependent cleavage of a tRNA which has a stem loop/hairpin structure.

Thus, in preferred embodiments, the polynucleotide comprises tRNA-derived polynucleotide fragment that has 14 to 35 nucleotides (tsRNA). tRNA-derived sRNAs are termed tsRNAs. The present inventor has shown that processing of tRNAs by the endoribonuclease Dicer, a critical player in canonical RNAi, results in the formation of tsRNAs. Without wishing to be bound by theory, it is the understanding of the inventor that Dicer associates with actively transcribed tRNA genes, binds to tRNAs that are folded into a non-canonical secondary structure (e.g. a structure that can be described as a stem-loop or short hairpin structure) and processes them into tsRNAs.

The present inventor has shown that tsRNAs are highly effective at binding the intronic regions of genes or long non-coding RNA to which they have complementarity and thereby inhibit expression of said gene or long non-coding RNA.

For example, the tsRNA molecule comprises between 14 to 25, 18 to 23, 20 to 22 or 25 to 28 nucleotides. In one embodiment, the polynucleotide comprises 18, 19, 20, 21, 22, 23, 24, 25, 26, 27 or 28 nucleotides. The tsRNA may be double stranded or single stranded. The double stranded tsRNA may be blunt ended. In another embodiment, the tsRNA is double stranded and tsRNA comprises an overhang. The tsRNA may be a mammalian, plant or bacterial tsRNA. For example, the tsRNA may be a human or rodent tsRNA. In one embodiment, the tsRNA is UA rich at the 5′ or 3′ end.

In one embodiment, the tsRNA, is chemically modified. Modifications can be introduced to promote stability, minimize innate immunity, and enable delivery to target tissues.

For example, at least one 2′-hydroxyl group of the nucleotides of the ds or ss tsRNA can be replaced by a chemical group, preferably a 2′-amino or a 2′-methyl group. At least one nucleotide in at least one strand can also be a locked nucleotide with a sugar ring which is chemically modified, preferably by a 2′-O, 4′-C methylene bridge. Advantageously, several nucleotides are locked nucleotides. Phosphorothioate modification can also be included. Other modifications are the inclusion of DNA or RNA analogues, such as inverted dT or abasic site. Or conjugates such as a peptide nucleic acid conjugate.

For example, single stranded tsRNA might be degraded by nucleases. However, this can be eliminated by modifying tsRNA ends either by adding a chemical modification or by locking the sugar bonds (LNA).

In one embodiment, the tRNA-derived polynucleotide is conjugated to another moiety, for example an N-acetylgalactosamine (GalNAc) to aid delivery into the nucleus.

The inventor has shown that a tsRNA with a specific sequence can target more than one gene. This can be beneficial if targeting of more than one gene in one pathway is required. In other example, it may be desirable to make tsRNA more specific so that only one gene is targeted. This can be achieved by adding several nucleotides from the target gene sequence (i.e. more than 5 residues) on each side of tsRNA.

The isolated tRNA-derived polynucleotide, e.g. the tsRNA, comprises a sequence that is complementary to an intronic region of the target gene or of long non-coding RNA. The tRNA-derived polynucleotide inactivates the target gene or long non-coding RNA through nascent RNA degradation. Furthermore, the tsRNA-derived polynucleotide is not derived from mature tRNA. It is derived from a species of aberrantly folded tRNA that forms a stem loop/hairpin structure and is cleaved by a Dicer dependent mechanism. tsRNA as described herein are capable of targeting nascent RNA in the nucleus. tsRNA target the intronic regions of specific genes to downregulate their expression through cleaving nascent RNA in an Ago2-dependent manner.

The skilled person working in this technical field will readily appreciate that an intron is a segment of DNA or RNA or a spliced regions of a non-coding RNAs which does not code for proteins and typically interrupts the protein-coding regions of genes (exons). In the present invention, the polynucleotide is complementary to an intronic region of the gene whose expression is to be inhibited. The skilled person will appreciate that it is not necessary for the entirety of the polynucleotide to be complementary to the intronic region of the gene. However, the polynucleotide should be sufficiently complementary that the polynucleotide binds to and/or hybridises to the intronic region of the gene.

As used herein, complementary is used to mean substantially complementary. In other words, complementarity does not have to be 100%. “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%», and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence.

Thus, in some embodiments, the polynucleotide comprises a sequence that is at least 50%, 60%, 70%, 80%, 90% or 95% complementary to a sequence of an intronic region of the gene. Preferably, the polynucleotide may comprise a sequence that is at least 95%, 96%, 97%, 98% or 99% complementary to a sequence of an intronic region of the gene.

In preferred embodiments of the present invention, the polynucleotide binds and/or hybridises to an intronic region of the mRNA of the gene whose expression is to be inhibited. In embodiments, the polynucleotide binds an intronic region of the nascent mRNA of the gene whose expression is to be inhibited. The present inventor has surprisingly found that by utilising a tRNA-derived polynucleotide which is complementary to, and can therefore bind to, an intronic region of the mRNA of the gene whose expression is to be inhibited, highly efficient inhibition of the expression of said gene can be achieved. Without wishing to be bound by theory, the inventor proposes that this is achieved by degradation of the nascent RNA. Thus, the invention also relates to methods as set out below.

Preferably, the target gene is associated with a pathological condition. The inventor has surprisingly found that the majority of genes that are targeted by tsRNA are disease associated genes, in particular genes that, when overexpressed, are associated with a disease phenotype. In one embodiment, the target gene is associated with cancer. In one embodiment, the target gene is associated with a disease as shown in FIG. 10, 11 or 12. In one embodiment, the target gene is selected from a gene of FIG. 10, 11 or 12. In one embodiment, the target gene is selected from the following human genes or long non-coding RNA: epidermal growth factor receptor (EGFR, see e.g. UniProtKB: P00533), MET (MET Proto-Oncogene, Receptor Tyrosine Kinase, see e.g. UniProtKB: P08581), BCL2 (BCL2 Apoptosis Regulator, see e.g. UniProtKB: P10415), LINC0665 (long intergenic non-coding RNA 00665, Lieu et al, Mol Ther Nucleic Acids. 2019 Jun. 7; 16: 155-161) or LIN000660. Exemplary tsRNA sequences to target these genes are shown in the examples and within the scope of the invention. The pathological condition may be selected from cancer, autoimmune diseases, neurodegenerative diseases such as Alzheimer's disease or Parkinson's disease, metabolic diseases, respiratory diseases and cardiovascular diseases. In one embodiment, the pathological condition is selected from one of the conditions as shown in FIG. 10, 11 or 12.

In one embodiment, the tsRNA has a sequence comprising SEQ ID NO. 4, 5 or 6.

In another aspect, we provide a vector comprising an isolated tRNA-derived polynucleotide, such as a tsRNA fragment as described above. Also provided is a host cell comprising the tRNA-derived polynucleotide, such as a tsRNA fragment or the vector as described above. The host cell may be mammalian, viral, bacterial, a plant or yeast cell. The vector may be a plasmid or a viral vector. Methods such as injection of naked tsRNA, physical delivery such as electroporation, gene gun, sonoporation, magnetofection, hydrodynamic delivery and chemical methods to enhance delivery such as inorganic nanoparticles and cell-penetrating peptides can be used for delivery.

Methods

The present invention provides a method of inhibiting or downregulating expression of a gene in a biological system. Inhibition or downregulation of expression is achieved by introducing a tRNA-derived polynucleotide, e.g. a tsRNA, which is complementary to an intronic region of the gene, into the biological system.

Thus, provided is a method of inhibiting or downregulating expression of a target gene or non-coding RNAs in a biological system, the method comprising:

introducing a tRNA-derived polynucleotide e.g. a tsRNA, into the biological system, wherein the polynucleotide comprises a sequence that is complementary to an intronic region of the target gene or non-coding RNAs.

The tRNA-derived polynucleotide is as described above, for example a tsRNA as described above. In one embodiment, the tRNA-derived polynucleotide is comprised in a vector as described above.

As will be appreciated by the skilled person, the phrase “inhibiting expression of a gene” does not necessarily require that the expression of the gene be entirely silenced. In embodiments, the method may result in substantially complete inhibition of expression of the gene or non-coding RNA (i.e. 100% inhibition or near 100% of gene expression). However, in alternative embodiments, the method of the present invention may result in partial, e.g. a slight or moderate reduction in the expression of the target gene or non-coding RNA.

For example, the method can result in expression of the gene or non-coding RNA being inhibited/downregulated by at least 10%, 20%, 30%, 40% or 50% compared to normal or wildtype expression. As would be appreciated by a skilled person, the amount of inhibition required is dependent on the gene targeted. For example, the method of the present invention results in expression of the gene being inhibited/downregulated by at least 60%, 70%, 80%, 90%, 95%, 98% or 99% compared to normal or wildtype expression.

The method results in inhibition of expression of a gene or non-coding RNA in a biological system. However, the skilled person will appreciate that the method may result in simultaneous inhibition of expression of multiple genes. In such embodiments, inhibition of expression of said multiple genes is achieved by introducing a tRNA-derived polynucleotide which is complementary to intronic regions of said multiple genes.

The method of the present invention may be used to inhibit expression of any target gene, that is any gene of interest, or any target non-coding RNA, provided a tRNA-derived polynucleotide, e.g. a tsRNAs as described herein, complementary to an intronic region of that gene is available/can be designed for introduction into the biological system.

The method of the present invention may be used for research purposes, for example to determine the function of a particular gene or the effect of reducing expression of that gene. In such embodiments, the gene may be a gene regarding which further information is sought. The gene may, in certain embodiments, be a gene associated with a particular disease. For example, in embodiments of the present invention in which the method is used for research purposes, a gene may be purportedly disease-linked, and the method may be used to determine the effect of inhibition of that gene in an in vitro, in vivo, ex vivo or in silico system.

For example, as described above, the gene is a gene associated with a disease, for example cancer, autoimmune diseases, neurodegenerative diseases such as Alzheimer's disease, viral or bacterial infectious diseases etc.

Biological System

The method described above results in inhibition/downregulation of expression of a gene or of a long non-coding RNA in a biological system. The method comprises introducing a tRNA or tRNA-derived polynucleotide e.g. a tsRNA, into the biological system.

As will be appreciated by the skilled person, the biological system of the present invention can be any biological system comprising a gene whose expression is to be inhibited. For example, the biological system may comprise a cell or plurality of cell, for example a eukaryotic cell/cells. The sample may include a cell, tissue, blood, urine, saliva, exosome, CSF or other sample from a human or animal, such as a mammal, subject. In another example, the sample may include a cell or tissue from a plant. In certain embodiments, the biological system may be a human or animal subject, for example a subject in which inhibition of gene expression is required. In embodiments, the biological system may comprise a synthetic biological system, created from component parts in vitro or created in silico, for example.

The method of the present invention may be performed in vitro. For example, in such an embodiment, the biological system may comprise a cell, tissue, blood sample or other sample from a human, plant or animal subject. In such embodiments, inhibition of gene expression may be required for research purposes, for example in order to determine the function of said gene or to determine whether inhibition of said gene results in a particular outcome.

Alternatively, the method of the present invention may be performed in vivo. For example, in such an embodiment, the biological system may be a human, plant or animal subject. In such embodiments, inhibition of gene expression may result in an altered phenotype in said human or animal subject. For example, inhibition of gene expression, where the gene is disease linked, may result in treatment of that disease.

In yet further embodiments, the method of the present invention may be performed ex vivo.

The method described above of the present invention involves introducing a tRNA-derived polynucleotide e.g. a tsRNA, into the biological system. The skilled person will appreciate that there are numerous methods by which the tRNA-derived polynucleotide may be introduced into the biological system. Such exemplary methods may include, for example, the use of non-viral vectors such as exosomes, nanoparticles or liposomes (e.g. lipofectamine) or viral vectors for example retroviral vectors, adenoviral vectors or herpes simplex viral vectors.

Other non-limiting delivery methods may include injection of naked tsRNA, physical delivery such as electroporation, gene gun, sonoporation, magnetofection, hydrodynamic delivery and chemical methods to enhance delivery such as inorganic nanoparticles and cell-penetrating peptides. Other delivery methods known in the art for siRNA can also be used.

In embodiments of the invention in which the tRNA-derived polynucleotide comprises tRNA, the method may further comprise introducing an enzyme into the biological system which cleaves tRNA to produce tsRNA. In embodiments, the enzyme comprises Dicer.

The present inventor has shown that Dicer, a critical player in canonical RNAi, associates with actively transcribed tRNA genes, binds to tRNAs that are folded into a non-canonical secondary structure (a stem-loop structure) and processes them into tsRNAs. The present inventor has effectively used such tsRNAs to target and inhibit expression of genes.

The skilled person will appreciate that in some biological systems utilised in the present invention, Dicer will naturally be present. However, in certain systems, for example synthetic biological systems, Dicer may not be present and the introduction of Dicer into the biological system may be advantageous.

In embodiments, the method of the present invention may comprise introducing a tRNA-derived polynucleotide into the nucleus of a cell in the biological system. As the skilled person will appreciate, there are various methods by which a polynucleotide can be introduced into the nucleus of a cell as known in the art. These include, for example, pronuclear injection and other microinjection methods or targeting the polynucleotide to the nucleus using a nuclear targeting sequence operably linked to the polynucleotide.

In embodiments of the present invention, the method may further comprise introducing an enzyme into the biological system which transports tRNA-derived polynucleotides to the nucleus. In embodiments, the enzyme comprises Argonaute 2 (Ago2).

The present inventor has surprisingly shown that Ago2 associates with tsRNAs and shuttles the tsRNAs to the nucleus where they can inhibit expression of a gene by binding the intronic regions of nascent mRNA.

The skilled person will appreciate that in some biological systems utilised in the present invention, Ago2 will naturally be present. However, in certain systems, for example synthetic biological systems, Ago2 may not be present and the introduction of Ago2 into the biological system may be advantageous.

The skilled person will appreciate that there are numerous methods by which an enzyme, such as Ago2 or Dicer, may be introduced into the biological system. Such methods may include, for example, the use of non-viral vectors such as exosomes, nanoparticles or liposomes or viral vectors for example retroviral vectors, adenoviral vectors or herpes simplex viral vectors. Alternatively, expression vectors which will express the enzyme under certain conditions within the biological system may be utilised to introduce such enzymes. The use of such expression vectors will be well understood by the skilled person working in this technical field.

We also provide a method of degrading nascent RNA in a biological system, the method comprising:
introducing a tRNA-derived polynucleotide into the biological system, wherein the polynucleotide comprises a sequence that is complementary to an intronic region of the nascent RNA.
The method may further comprise isolating a tsRNA.

We also provide a method for identifying a tsRNA fragment that mediates RNA interference of a target gene said method comprising:

a) providing a sample;

b) isolating a tsRNA fragment having between around 14 and 35 nucleotides from said sample;

c) characterising the tsRNA fragment to determine sequence identity or similarity with the target gene and;

d) identifying a tsRNA fragment that comprises a sequence that is complementary to an intronic region of a target gene.

The sample may be tissue, cells, blood, serum, exosomes or another sample.

We also provide a method for producing a tsRNA that mediates RNA interference comprising identifying a tsRNA fragment according to the method above. This may involve synthesising tsRNA based on the sequence of the identified tRNA fragment.

We also provide a method for producing a tsRNA from a tRNA which has a stem loop/hair pin structure comprising cleaving the stem loop/hairpin structure. In a Dicer dependent manner.

Another aspect relates to a tsRNA fragment that mediates RNA interference obtained or obtainable by the method described above.

We also provide a method of mediating RNA interference, the method comprising: introducing a tRNA-derived polynucleotide, e.g. a tsRNA described herein into a biological system.

We also provide a method for mediating target specific RNA interference in a cell comprising contacting the cell with a tRNA-derived polynucleotide, e.g. a tsRNA as described herein.

In all the methods above, the tRNA-derived polynucleotide is as described herein and is a tsRNA as described herein.

We further provide a method for designing a tRNA-derived polynucleotide, e.g. a tsRNA, that comprises a sequence that is complementary to an intronic region of the target gene and is capable of inhibiting gene expression of the target gene said method comprising as shown in FIG. 4a.

We further provide a method for designing a tRNA-derived polynucleotide, e.g. a tsRNA, that comprises a sequence that is complementary to an intronic region of the target gene and is capable of inhibiting gene expression of the target gene said method comprising:
generating a knockout of dicer and ago2;
identifying the genes that are upregulated when dicer and ago2 are knocked out;
generating a database of candidate targets;
identifying the intronic regions of the candidate targets;
generating, in silico, a panel of complimentary tsRNA sequence and
validating the targets to confirm the generated tsRNAs bind to the intronic regions predicted.

Use of tRNA Derived Polynucleotide as Biomarkers

In another aspect, a tRNA derived polynucleotide as described herein e.g. a tsRNA, can be used as a biomarker to detect the presence of a disease.

In another aspect, we provide a method of detecting a disease, the method comprising;

a) Detecting the presence of a tsRNA that has 14 to 35 nucleotides and is complementary to an intronic region of a target gene or of a long non-coding RNA in a sample;

b) Quantifying the amount of the tsRNA present in the sample;

c) Comparing the amount of tsRNA present in the sample to a reference value and;

d) Assessing the presence of absence of the disease.

In one embodiment, the reference is the amount of tsRNA in healthy cells or diseased cells, serum or exosomes. In one embodiment, the disorder is selected from cancer, autoimmune diseases, neurodegenerative diseases, metabolic diseases, respiratory diseases and cardiovascular diseases. In one embodiment, the isolated tsRNA is quantified by methods known in the art, such as RT-PCR. In one embodiment, the sample is a blood sample. In one embodiment, the method is performed in vitro.

Medical Uses and Methods

The present invention also provides a tRNA-derived polynucleotide, e.g. a tsRNA, as described above comprising a sequence that is complementary to an intronic region of a gene or long non-coding RNA or a pharmaceutical composition as described herein, for use as a medicament.

There is also provided a tRNA-derived polynucleotide, e.g. a tsRNA, as described above comprising a sequence that is complementary to an intronic region of a gene or long non-coding RNA or a pharmaceutical composition as described herein, for use in treating a disease which can be at least partially ameliorated by inhibiting expression of the gene.

Suitably, the use involves administering to a subject with the disease a therapeutically effective amount of tRNA-derived polynucleotide.

The present invention also provides a method of treating a disease in a subject comprising administering to the subject a therapeutically effective amount of tRNA-derived polynucleotide, e.g. a tsRNA, as described above or of a pharmaceutical composition as described herein, wherein the disease can be at least partially ameliorated by inhibiting expression of the gene and the tRNA-derived polynucleotide comprises a sequence that is complementary to an intronic region of the gene or long non-coding RNA.

Features of the tRNA-derived polynucleotide, e.g. a tsRNA, are described above in relation to the first aspect of the invention. In one embodiment, the tRNA-derived polynucleotide is a tsRNA as described above.

As will be appreciated by the skilled person, there are many diseases which are linked to the expression of genes. For example, the expression or overexpression of certain genes can result in disease phenotypes. Examples include the overexpression of α-synuclein which is linked to the development of Parkinson's disease and the overexpression of p53 which is associated with various forms of cancer. Other diseases are listed in FIGS. 10, 11 and 12. As will be understood by the skilled person, when diseases result from the expression or overexpression of genes, such diseases can be treated by inhibiting expression of the gene. So, for example, by reducing expression of α-synuclein it may be possible to treat Parkinson's disease.

As will be appreciated by the skilled person, the terms “treating”, “treats” and “treatment” include both preventative and curative treatment of a condition, disease or disorder. These terms also include slowing, interrupting, controlling or stopping the progression of a condition, disease or disorder and preventing, curing, slowing, interrupting, controlling or stopping the symptoms of a condition, disease or disorder.

The present inventor has shown that by utilising a tRNA-derived polynucleotide which is complementary to an intronic region of a gene, expression of that gene can be inhibited. By targeting specific disease-associated genes (whose disease results from expression or overexpression of said gene), it is possible to treat the disease.

The disease treated by the tRNA-derived polynucleotide, e.g. a tsRNA, is a disease which can be at least partially ameliorated by inhibiting expression of a gene to which the tRNA-derived polynucleotide is complementary to an intronic portion. In particular embodiments, the disease may be selected from cancer, autoimmune diseases, neurodegenerative diseases such as Alzheimer's disease or Parkinson's disease, metabolic diseases, respiratory diseases and cardiovascular diseases, for example. Other diseases are listed in FIGS. 10, 11 and 12.

As will be appreciated by the skilled person, the phrase “inhibiting expression of a gene” does not necessarily require that the expression of the gene be entirely silenced. In embodiments, the tRNA-derived polynucleotide may completely inhibit expression of the gene (i.e. 100% inhibition of gene expression). However, in alternative embodiments, the tRNA-derived polynucleotide may result in a slight or moderate reduction in the expression of a gene.

Preferably, the tRNA-derived polynucleotide results in expression of the gene being inhibited by at least 50% compared to normal or wildtype expression. Preferably, the tRNA-derived polynucleotide results in expression of the gene being inhibited by at least 60%, 70%, 80%, 90%, 95%, 98% or 99% compared to normal or wildtype expression. Preferably the tRNA-derived polynucleotide, e.g. a tsRNA, results in complete inhibition of expression of the gene (i.e. expression of the gene being inhibited by 100%).

In the method of treating a disease, the method comprises administering a tRNA-derived polynucleotide, e.g. a tsRNA, to a subject. The subject may be any subject in need of treatment for a disease wherein the disease can be treated by inhibiting expression of a gene. In embodiments, the subject is a human or animal subject.

The therapeutically effective amount of tRNA-derived polynucleotide, e.g. a tsRNA, may be administered orally, topically, by inhalation, insufflation or parenterally. As the skilled person will appreciate, the tRNA-derived polynucleotide, e.g. a tsRNA, may be formulated, for example in a pharmaceutical composition, for the particular administration route. For example, formulations suitable for oral administration include tablets, lozenges, hard or soft capsules, aqueous or oily suspensions, emulsions, dispersible powders or granules, syrups or elixirs. Suitable formulations for topical use include, for example, creams, ointments, gels, or aqueous or oily solutions or suspensions. Suitable formulations for inhalation include, for example, as a fine powder or a liquid aerosol. Suitable formulations for administration by insufflation include, for example, a fine powder. Suitable formulations for parenteral administration include, for example, a sterile aqueous or oily solution for intravenous, subcutaneous, intramuscular or intramuscular dosing or as a suppository for rectal dosing.

As will be appreciated by the skilled person, the therapeutically effective amount of tRNA-derived polynucleotide, e.g. a tsRNA, will necessarily vary depending on the subject to be treated, the route of administration and the nature and severity of the disease to be treated.

We also provide a combination therapy comprising administration of a tRNA-derived polynucleotide described above or pharmaceutical composition described above and an anti-cancer therapy. The anti-cancer therapy may be radiotherapy, chemotherapy, RNAi therapy, gene therapy or treatment with a biological or small molecule drug. The tRNA-derived polynucleotide or pharmaceutical composition and the anti-cancer therapy may be provided at the same time in the same or a different medicament. Alternatively, the therapies may be provided as different medicaments administered sequentially. In one embodiment, the other therapy is radiotherapy and the combination therapy may provide at least additive effects. Thus, we also provide methods for enhancing the therapeutic effects of radiotherapy by co-administration of a tRNA derived polynucleotide as described herein.

Pharmaceutical Composition

The present invention also provides a pharmaceutical composition comprising a tRNA-derived polynucleotide as described herein, e.g. a tsRNA, and a pharmaceutically-acceptable diluent or carrier.

There is also provided a pharmaceutical composition comprising a tRNA-derived polynucleotide, e.g. a tsRNA, as described herein and a pharmaceutically-acceptable diluent or carrier, for use as a medicament.

There is also provided a pharmaceutical composition comprising a tRNA-derived polynucleotide, e.g. a tsRNA, comprising a sequence that is complementary to an intronic region of a gene and a pharmaceutically-acceptable diluent or carrier, for use in treating a disease which can be at least partially ameliorated by inhibiting expression of the target gene or long non-coding RNA.

Features of the tRNA-derived polynucleotide e.g. a tsRNA, are described above.

As will be appreciated by the skilled person, the terms “treating”, “treats” and “treatment” include both preventative and curative treatment of a condition, disease or disorder. These terms also include slowing, interrupting, controlling or stopping the progression of a condition, disease or disorder and preventing, curing, slowing, interrupting, controlling or stopping the symptoms of a condition, disease or disorder.

The pharmaceutical composition of the present invention may be administered orally, topically, by inhalation, insufflation or parenterally. As the skilled person will appreciate, the pharmaceutical composition may be formulated for the particular administration route. For example, formulations suitable for oral administration include tablets, lozenges, hard or soft capsules, aqueous or oily suspensions, emulsions, dispersible powders or granules, syrups or elixirs. Suitable formulations for topical use include, for example, creams, ointments, gels, or aqueous or oily solutions or suspensions. Suitable formulations for inhalation include, for example, as a fine powder or a liquid aerosol. Suitable formulations for administration by insufflation include, for example, a fine powder. Suitable formulations for parenteral administration include, for example, a sterile aqueous or oily solution for intravenous, subcutaneous, intramuscular or intramuscular dosing or as a suppository for rectal dosing.

The pharmaceutical composition of the invention may be obtained by conventional procedures using conventional pharmaceutical excipients. For example, pharmaceutical compositions intended for oral administration may contain, for example, one or more colouring, sweetening, flavouring and/or preservative agents.

As will be appreciated by the skilled person, the amount of active ingredient (i.e. tRNA-derived polynucleotide) will necessarily vary depending on the host treated and the route of administration. The amount of active ingredient will necessarily vary according to the nature and severity of the disease to be treated, the age and sex of the patient or animal and the route of administration.

The pharmaceutical composition may be specifically formulated for delivery of RNA molecules non-viral vectors such as exosomes, nanoparticles or liposomes or viral vectors for example retroviral vectors, adenoviral vectors or herpes simplex viral vectors or lipid conjugated. Other potential delivery methods are listed elsewhere herein.

Uses

The present invention also provides the use of a tRNA-derived polynucleotide as described herein, e.g. a tsRNA, for inhibiting expression of a gene or long non-coding RNA in a biological system, wherein the polynucleotide comprises a sequence that is complementary to an intronic region of the gene.

In some embodiments, the use is an in vitro use i.e. a non-medical use. In other embodiments, the use is an ex vivo use, for example use in conditioning cell therapies.

Kit

The present invention also provides a kit comprising the tRNA derived polynucleotide as described herein, e.g. a tsRNA.

Methods for Designing Molecules

We also provide a computer implemented method for generating a candidate tRNA-derived polynucleotide, e.g. a tsRNA, that comprises a sequence that is complementary to an intronic region of the target gene as described herein and is capable of inhibiting gene expression of the target gene said method comprising:

a) Determining the inherent features of the tRNA from which the said polynucleotide is derived;

b) Determining the inherent features of the binding sites within an intronic region of a target gene, to which the tRNA-derived polynucleotide binds;

c) Generating a dataset comprising known tRNA-derived polynucleotide and binding sites;

d) Using the dataset to define a training dataset to identify any patterns in structure, nucleotide content, position within an intron, primary, secondary or tertiary structure or gene targets;

e) Screening a genome sequence using the training dataset to identify candidate binding sites within intronic region of the target gene and;

f) Generating a candidate tRNA-derived polynucleotide that comprises a sequence that is complementary to an intronic region of the target gene.

In one embodiment, the inherent features include sequence, secondary structure, and/or location within the genome.

We also provide a computer system for identifying one or more unique tRNA-derived polynucleotide sequences in a genome of a eukaryotic organism, the system comprising:

I. a memory unit configured to receive and/or store sequence information of the genome; and

II. one or more processors alone or in combination programmed to perform a method described above.

Features of the use of the present invention are described in further detail in relation to the abovementioned aspects of the invention, in the examples below.

The described and illustrated examples are to be considered as illustrative and not restrictive in character, it being understood that only the preferred embodiments have been shown and described and that all changes and modifications that come within the scope of the inventions as defined in the claims are desired to be protected.

It should be understood that while the use of words such as “preferable”, “preferably”, “preferred” or “more preferred” in the description suggest that a feature so described may be desirable, it may nevertheless not be necessary and embodiments lacking such a feature may be contemplated as within the scope of the invention as defined in the appended claims. In relation to the claims, it is intended that when words such as “a,” “an,” or “at least one,” are used to preface a feature there is no intention to limit the claim to only one such feature unless specifically stated to the contrary in the claim.

### FIGURES

The present invention will now be further described with reference to the following non-limiting figures which show:

FIG. 1: Dicer associates with tRNA genes, binds alternatively folded tRNAs and processes them into tsRNAs. A) Heatmaps showing ChIP-seq data. tRNA genes (Rows) are ranked according to RNAPIII occupation. B) Stacked bar charts representing the proportion of tRNAs that are (and not) associated with Dicer and RNAPIII. C) Northern blot iamge on which specific tRNA^{Arg-CCG-2-1}, tRNA^{Gly-CCC-2-1} and tRNA^{Pro-TGG-3-3} were probed in wildtype and shDicer cells. Schematic diagrams representing the clover-leaf and short hairpin structures corresponding to the bands. D) Northern blot image on which tRNA^{Gly-CCC-2-1} was probed in wildtype and shDicer. E) Boxplots of sRNA changes within tRNAs, miRNAs and snoRNAs between wildtype and shDicer cells. Dotted line is drawn across zero.

FIG. 2: Dicer associates with transcribed tRNA genes which can fold into short hairpin structures. A) Pie charts showing the proportion of tRNA genes that are associated (and not) with Dicer and RNAPIII. B) Schematic diagrams representing the secondary structures formed by tRNA^{Arg-CCG-2-1} and tRNA^{Gly-CCC-2-1}. dG indicates free energy. C) Western blot images showing the levels of RNAPII, RNAPIII and Dicer upon shDicer (β-tubulin as loading control).

FIG. 3: Analysis of tsRNA biogenesis and origin. A) sRNA levels in shDicer (D) vs normal cells (N) grouped by absolute difference D−N>0 and D−N<0. B) Number of tsRNAs mapping to each tRNA position (given in % of tRNA length).

FIG. 4: Dicer-dependent tsRNAs are predicted to target introns of genes without affecting chromatin state. A) Diagram depicting the bioinformatics workflow for predicting genes targeting by Dicer- and Ago-associating tsRNAs. B) Bar chart showing exon-based qRT-PCR analysis of six selected targeted genes upon shDrosha, shDicer and shAgo2 respectively. C) Bar chart showing intron-based qRT-PCR analysis of six selected target genes upon shDicer and shAgo2 respectively. D-F) Bar charts showing ChIP analyses of total and active RNAPII levels upon shDicer across three regions of SPINT1. Mean±s. d. (n=3, *P<0.05) are shown.

FIG. 5: Target genes were upregulated in both cytoplasm and nucleus. A) Western blot images showing levels of Dicer and Drosha in wildtype, shDicer and shDrosha cells (p63 as loading control). B) Western blot images showing levels of Dicer and Ago2 in wild type, shDicer and shAgo2 cells (Ponceau S as loading control) C) Bar charts showing levels of NOV, GUCY1A2, GK and RBP7 in cytoplasmic and nuclear fractions in wildtype and shDicer cells. Mean±s. d. (n=3) are shown.

FIG. 6: tsRNAs do not lead to transcriptional gene silencing. A) Bar charts showing ChIP analyses of total and active forms of RNAPII upon shDicer across GK and GUCY1A2. Mean±s. d. (n=3) are shown. B) Bar chart showing ChIP analysis of H3K9me2 upon shDicer at loci of target genes. Mean±s. d. (n=3) are shown. C) Western blot images showing levels of total and active RNAPII, H3 and H3K9me2 upon shDicer (β-tubulin as loading control).

FIG. 7: ChrRNA-seq analysis of tsRNA targets. A) Bar charts showing qRT-PCR analyses of DROSHA, DICER and AGO2 transcript levels at the chromatin upon shDrosha, shDicer and shAgo2, respectively, in two biological replicates subjected to chrRNA-seq. B) Schematic diagram depicting the workflow for predicting target genes using chrRNA-seq data followed by disease-gene association analysis.

FIG. 8: Presence of active epigenetic marks and absence of repressive marks on SPINT1. H327Ac, H3K4me3 and H3K9me3 profiles across SPINT1 gene. Read counts are indicated on the left.

FIG. 9: ChrRNA-seq analysis of target genes and validation of tsRNA medicated gene silencing. A) Venn diagrams showing the upregulated genes in chrRNA-seq (P<0.005 for shDicer and shAgo2, P<0.05 for shDrosha). B) chrRNA-seq profiles across SPINT1 (n=2). Normalised read counts are indicated in brackets. C) Pie chart representing the proportion of upregulated genes targeted by tsRNAs across different gene regions. D) Pie chart representing the proportion of tRNAs that have targets in different gene regions. E) Bar chart (top) representing qRT-PCR analysis of GK and SPINT1 mRNA upon shDicer with and without transfection of specific tsRNA. Means±s. d. (n=4) are shown. Schematic diagram representing the region targeted by the tsRNA and its origin. F) Western blot images showing Ago2 levels in cytoplasmic and chromatin fractions of cells with and without treatment of aAmanitin. G) Schematic diagram depicting model for gene silencing mechanism.

FIG. 10: Genes silencing via Dicer-dependent tsRNAs-mediated nascent RNA degradation are associated with various diseases. A) Contingency table of target and disease-associated genes (P<2.2×10⁻¹⁶, one-sided Fisher's exact test). B) Heatmap, sorted bi-directionally for gene-disease associations, of top 50 diseases against top 100 target genes. Blue indicates a match while white indicates no match. C) Stacked bar chart showing disease categories related to the 100 top diseases that are associated with target genes. 63 disease are cancer, 17 are nervous system diseases and so forth.

FIG. 11: tsRNAs are associated with various diseases. Heatmap, sorted bi-directionally for gene-disease associations, of top 100 diseases against top 100 target genes. Blue indicates a match while white indicates no match.

FIG. 12: tsRNAs are associated with various disease classes. Heatmap, sorted bi-directionally for gene-disease associations, of top 100 disease classes against top 100 target genes. Blue indicates a match while white indicates no match.

FIG. 13: A) Steady state levels of EGFR and MET mRNA were reduced upon transfection of tsRNA EGFR/MET. B) Nascent levels of EGFR and MET mRNA were reduced upon transfection of tsRNA EGFR/MET. Bars from left to right represent: BT−; BT tsRNAEGFR/MET; BT− tsRNASPINT1.

FIG. 14: BT549 Cells were transfected with tsRNA EGFR/MET and imaged on day 3 using a light microscope. More cells appeared to be dead in the population of cells transfected with tsRNA EGFR/MET.

FIG. 15: Number of dead cells increased with increasing amount of tsRNA EGFR/MET (light microscopy).

FIG. 16: Number of live cells decreased with increasing amount of tsRNA EGFR/MET (crystal violet staining).

FIG. 17: Both transfections of tsRNA EGFR/MET and siRNA targeting EGFR led to downregulation of EGFR and MET but not control genes with higher efficacy with siRNA. A) steady state mRNA after 24 hours; B) steady state mRNA levels after 72 hours. C) Western blot showing protein levels after 24 hours; D) Western blot showing protein levels after 72 hours. Bars from left to right represent: BT−; BT ts; BTsi

FIG. 18: Transfection of tsRNA BCL2 into cells led to downregulation of steady state BCL2 mRNA levels. Bars from left to right represent: MCF7−; MCF7 tsRNABCL2; MCF7 tsRNASPINT1

FIG. 19: Western blot showing BCL-2 levels decreased with increasing amount of tsRNA BCL2 transfected. Cleaved Caspase-9 increased with increasing amount of tsRNA BCL2, indicating more cells were undergoing apoptosis.

FIG. 20: MCF7 cells were transfected with tsRNA BCL2 and imaged on day 3 with light microscopy. More dead cells were present in population of cells transfected with tsRNA BCL2.

FIG. 21: Fewer live cells were present in populations of cell transfected with tsRNA BCL2.

FIG. 22: LINC0665 levels were reduced by transfection of tsRNA LINC0665 in BT549 cells. Bars from left to right represent: A−; tsRNALINC0665; tsRNASPINT1.

FIG. 23: Both steady state (A) and nascent (B) LINC0665 levels were reduced by transfection of tsRNA LINC0665 in A549 cells. Bars from left to right represent: A−; tsRNALINC0665; tsRNASPINT1.

FIG. 24: A549 cells were transfected with tsRNA LINC00665 and imaged on day 3 with light microscopy. More dead cells were present in population of cells transfected with tsRNA LINC00665.

FIG. 25: Fewer live cells were present in populations of cells transfected with tsRNA LINC00665.

FIG. 26: Irradiated cells showed higher gamma-H2AX signals 30 mins post-irradiation. However, at the 90-min time point, cells transfected with ts20 (i.e. tsRNA targeting LINC00665) failed to repair DNA damage like the controls, suggesting that tsRNA LINC00665 is affecting genes involved in DNA damage response.

FIG. 27: Transfection of tsRNA LINC00665 into MCR7 and BT549 cells caused cell death; more cell death occurred when the cells were subjected to gamma-irradiation. However, this effect was magnified when transfection of tsRNA LINC00665 was combined with gamma-irradiation. A) Cells exposed to 0 Gy; B) cells exposed to 10 Gy.

FIG. 28: Transfection of tsRNA BCL2 into MCR7 and BT549 cells caused cell death; more cell death occurred when the cells were subjected to gamma-irradiation. However, this effect was magnified when transfection of tsRNA LINC00665 was combined with gamma-irradiation. A) Cells exposed to 0 Gy; B) cells exposed to 10 Gy.

FIG. 29: a. Native PAGE showing in vitro transcribed RNA of tRNA^{Arg-CCG-2-1} and miRNA pre-let7a. Schematic diagrams representing the clover-leaf and short hairpin structures corresponding to the bands. B. In vitro transcribed miRNA pre-let7a, tRNA^{Arg-CCG-2-1} and snoRD38A were incubated with purified Dicer-TAP. Aliquots were taken at time points as indicated and analysed on denaturing PAGE. C. Northern blot analysis of in vitro transcribed tRNA^{Arg-CCG-2-1} incubated in presence of purified Dicer-TAP at 0, 30, 90 and 240 min.

FIG. 30: a. Combined snapshot of RNAP II ChIP-seq, RNAP II mNET-seq and chrRNA-seq profiles in wt HEK293 cells and Drosha, Dicer and Ago2 knockdowns across target gene RP4-639F20.1 (n=2). Normalised read counts are indicated in brackets. B. Combined snapshot of RNAP II ChIP-seq, RNAP II mNET-seq and chrRNA-seq profiles in wt HEK293 cells and Drosha, Dicer and Ago2 knockdowns across non-target gene GAPDH (n=2). Normalised read counts are indicated in brackets.

FIG. 31: a. Metagene representing the distribution of tsRNAs targeting introns FIG. 32: a Combined snapshot of RNAP II ChIP-seq, RNAP II mNET-seq and chrRNA-seq profiles in wt HEK293 cells and Drosha, Dicer and Ago2 knockdowns across target gene SPINT1 (n=2). Normalised read counts are indicated in brackets. b. Combined snapshot of RNAP II ChIP-seq, RNAP II mNET-seq and chrRNA-seq profiles in wt HEK293 cells and Drosha, Dicer and Ago2 knockdowns across target gene GK (n=2). Normalised read counts are indicated in brackets.

FIG. 33a Confocal images showing localization of in vitro fluorescently (green) labelled siRNA and tsRNA targeting EGFR target gene. Nuclei were stained in blue.

FIG. 34: a. Northern blot showing RNA samples isolated from fractionated cells: WC whole cells, C cytoplasmic fraction and N nuclear fraction. Signals are shown for two tRNAs. Region of small RNA is depicted by vertical line on the right. b. Northern blot showing signal for specific tsRNA bound to Ago2. IgG was used as negative control.

FIG. 35a In vitro cleavage assay. Full length substrate (part of SPINT1 intron) containing target site was incubated with purified Ago2 followed by Northern blot. Position of the probe is depicted in red.

FIG. 36: a. Schematic diagram showing position of tsRNA targeting intron of SPINT1 gene. qRT-PCR showing levels of target SPINT1 RNA in wt cells transiently transfected with synthetic tsRNA. Mock was used as control. b. qRT-PCR showing levels of target SPINT1 RNA in wt and Dicer kd (shDicer) cells transiently transfected with synthetic tsRNA targeting SPINT1 and GK (negative control) genes. Mock was used as control. The 4 bars from left to right represent: wt+mock; shDicer+mock; shDicer+tsRNASPINT1; shDicer+tsRNAGK. c.qRT-PCR showing levels of target SPINT1 RNA in wt cells transiently transfected with different amounts of single stranded synthetic tsRNA targeting SPINT1. Mock was used as control. The 3 bars from left to right represent: wt_mock; wt+ss tsRNASPIN1 50 nM; wt+ss tsRNA SPIN 100 nM. d. qRT-PCR showing levels of target SPINT1 RNA in wt cells transiently transfected with different amounts of double stranded synthetic tsRNA targeting SPINT1. Mock was used as control. The 3 bars from left to right represent: wt_mock; wt+ds tsRNASPINT1 30 nM; wt+ds tsRNA SPIN 60 nM

As discussed above, the aspects of the present invention allows expression of a gene to be efficiently inhibited through use of a tRNA-derived polynucleotide which is complementary to an intronic region of the gene. The present inventor undertook significant investigation to develop the aspects of the present invention as described below.

### EXAMPLES

The invention is further described with reference to the following non-limiting examples.

### Example 1

Materials and Methods

Cell Lines and Treatments

The cell lines used in the studies undertaken by the present inventor were human embryonic kidney 293 (HEK293 cells), HEK293 clone 1.3 cells with integrated doxycycline-inducible expression cassettes containing TAP-tagged Dicer together with shRNA against DICER mRNA (shDicer) and HEK293-based cell lines with integrated doxycycline-inducible expression cassettes containing shDicer and shRNA against AGO2 mRNA (shAgo2) respectively. All cells were cultured in Dulbecco's Modified Eagle Medium (DMEM) (Thermo Fisher Scientific) with 10% foetal bovine serum, 1% L-glutamine (Thermo Fisher Scientific) and 1% Penicillin-Streptomycin (Thermo Fisher Scientific) in 5% CO₂ at 37° C. Dicer and Ago2 knockdown were achieved by incubating the inducible cell lines with doxycycline (3 μg/ml) in DMEM for 72 hours (replaced with fresh media with doxycycline every 24 hours) at 37° C. TAP-tag in HEK293T clone 1.3 cells were induced with doxycycline (3 μg/ml) for 5 days. Transfection of shRNA against DROSHA mRNA (shDrosha)-containing plasmids (10 μg for 6 hours twice) and tsRNA (50 μM for 48 hours) were performed using Lipofectamine 2000 reagent (Invitrogen). Cell were incubated with α-Amanitin (2 μg/ml, Sigma) for 24 hours to inhibit transcription.

Northern Blot

2 to 3 μg of RNA in 2× native loading dye (0.05% xylene cyanol, 0.05% bromophenol blue, 20% glycerol) was separated on 14% bis-polyacrylamide gels in 1×TBE followed by transfer onto nitrocellulose membrane (Protran, GE Healthcare). Membranes were UV-crosslinked and pre-hybridised in oligo hybridisation buffer at 42° C. for 1 hour. tRNA-specific oligonucleotide probes were radiolabelled with ₃₂P-ATP by polynucleotide kinase (PNK) at 37° C. for 30 minutes. Radiolabelled probes were purified in G-25 Sephadex columns (GE Healthcare) and hybridised onto the membrane O/N at 42° C. followed by washes with Northern wash buffer (0.05% SDS, 0.1×SCC) and subjected to autoradiography.

Western Blot

Whole cell, cytoplasmic, nuclear or chromatin extracts were treated directly with 4× Laemmli buffer (0.2 M Tris-HCl, 8% (w/v) SDS, 40% glycerol, 20% (v/v) β-mercaptoethanol, 0.005% bromophenol blue), incubated at 95° C. for 5 minutes and sonicated. Samples were separated on mini-PROTEAN® TGX™ gels (Bio-Rad Laboratories) followed by transfer onto nitrocellulose membranes (Protran, GE Healthcare) and probed with antibodies.

Preparation of sRNA-Seq and mRNA-Seq Samples

For sRNA-seq, total RNA was isolated from cells treated with scrambled shRNA (as control) and shDicer cells for 7 days using the miRVana miRNA Isolation Kit (Thermo Fisher Scientific). Quality of purified RNA was confirmed with RNA 6000 Pico Kit (Agilent) on the Agilent 2100 Bioanalyzer. Sequencing libraries were prepared using the NEBNext® Multiplex Small RNA Library Prep Set (New England BioLabs) and sequenced on a HiSeq2000 (Illumina). For mRNA-seq, RNA was purified using the miRNEasy Kit (Qiagen) and treated with DNase (Thermo Fisher Scientific) at 37° C. for 30 minutes followed by acidic phenol-chloroform extraction. Sample integrity was verified with a 1.25% formaldehyde gel. RNA samples were ribo-depleted and sequencing libraries preparation was performed with the TruSeq Stranded Total RNA Sample Preparation Kit (Illumina) followed by paired-end sequencing on HiSeq2000 (Illumina).

Reverse Transcription-Quantitative PCR

RNA was isolated from whole-cell, cytoplasmic, nuclear or chromatin extracts with TRIzol (Invitrogen) as per manufacturer's instructions and treated with DNase I (1 U, Roche) for 30 minutes at 37° C. 250-500 ng (exon-based) and 5 μg of RNA (intron-based) were used for preparing cDNA template using SuperScript™ Reverse Transcriptase (Thermo Fisher Scientific) with specific reverse primers. Real-time PCR was performed on Rotor-Gene RG3000 machine (Corbett Research) with SensiMix™ SYBR No-Rox Mastermix (Bioline Reagents) with specific primer pairs. Relative fold change was computed using the comparative Ct method (1).

Subcellular Fractionation

Cytoplasmic and nuclear fractions were obtained according to a published protocol (2).

Chromatin-Associated RNA Sequencing

Chromatin fraction was extracted using approximately 6.72×10⁶ cells according to a published protocol (3) and treated with 40 μg of proteinase K in 1% SDS and 1 μl of Turbo DNase (2 U/μl) (Thermo Fisher Scientific), which was followed by TRIzol (Invitrogen) extraction. Incompletely dissolved chromatin pellet was dissolved by heating the samples at 55° C. for 10 minutes on a heat block in safe lock tubes (Eppendorf).

Chromatin Immunoprecipitation

Approximately 7×10⁶ cells were incubated with 1% formaldehyde in DMEM for 8 minutes followed by quenching with 0.125 M glycine in DMEM for 10 minutes at 37° C. Cells were washed with ice-cold PBS and lysed in 500 μl cell lysis buffer (0.5% NP-40, 85 mM KCl, 5 mM PIPES, 1× protease inhibitor cocktail (Roche)). Chromatin was pelleted at 800 g for 10 min and lysed in nuclear lysis buffer (50 mM Tris-HCl, 1% SDS, 10 mM EDTA, 1× protease inhibitor cocktail (Roche)) and sonicated at high power settings for 25 min at 4° C. The fragmented chromatin lysate was pre-cleared with protein G magnetic beads (40 μl per sample, Invitrogen) for 1 hour, divided equally into input, IP and beads only samples and diluted in dilution buffer (16.7 mM Tris-HCl, 0.01% SDS, 1.1% Triton X-100, 500 mM EDTA, 167 mM NaCl, 1× protease inhibitor cocktail (Roche)). Immunoprecipitation with antibodies was performed overnight and samples were incubated with protein G magnetic beads (40 μl) for 1 hour. Beads were washed with washing buffers A (20 mM Tris-HCl, 2 mM EDTA, 0.1% SDS, 1% Triton X-100, 150 mM NaCl), B (20 mM Tris-HCl, 2 mM EDTA, 0.1% SDS, 1% Triton X-100, 500 mM NaCl), C (10 mM Tris-HCl, 1 mM EDTA, 1% NP-40, 1% sodium deoxycholate, 0.25 M LiCl) and D (10 mM Tris-HCl, 1 mM EDTA). Protein-DNA complexes were eluted with elution buffer (1% SDS, 0.1 M NaHCO₃) for 30 min at room temperature and treated with RNase A (1 μl) and proteinase K (2 μl) at 65° C. overnight. DNA was extracted with phenol-chloroform (1:1) mix and used followed by qPCR.

Statistical Analyses of Experimental Data

qPCR data were analysed using raw Ct values. When comparing two conditions, data were subjected to Shapiro-Wilk test and F test to assess for normality and equal variance; if they follow a normal distribution and have the same variance, unpaired t-test (one-tailed) was performed to test for significant difference (p-value <0.05 is considered as significant). If data do not follow a normal distribution, unpaired Mann Whitney test (one-tailed) was used instead. For comparison of two or more conditions, one-way ANOVA was performed followed by Tukey multiple comparisons test.

Bioinformatics Analyses

ChIP-seq

The inventor previously published Dicer ChIP-seq was analysed (4) (GSM1366345). Raw data was adapter trimmed with cutadapt 1.8.3 (5) for various contaminating sequences identified by fastqc (6). Thus AGATCGGAAGAGCTCGTATGCCGTCTTCTGCTTG (SEQ ID NO. 1), TCGTATGCCGTCTTCTG (SEQ ID NO. 2) and CTGTAGGCACCATCAAT (SEQ ID NO. 3) were trimmed at 3′ and 5′ ends.

RNAPIII ChIP-seq data was downloaded from GEO (GSM509047) (7), as well as RNAPII ChIP-seq data (GSM935534) and input (GSM935533) (ENCODE Transcription Factor Binding Sites by ChIP-seq from Stanford/Yale/USC/Harvard) (8). All ChIP-Seq data was mapped to hg38 using bowtie2 (9) with default values. Reads with samflag 4 (unmapped) were discarded and duplicate reads were removed using samtools 0.1.19 (10). Bedgraphs were generated by using bedTools genomeCoverageBed (11) and normalizing by (library size)/10⁸. Presence of RNAPIII and Dicer was determined by peak-calling with MACS version 2.1.1 (12) and command line arguments callpeak -g hs --broad-cutoff 0.05 -broad.

tRNA hg38 coordinates were downloaded from UCSC (13). Coverage values for each tRNA and the 200 nt surrounding region were computed with a custom written perl script. Subsequently each tRNA was stretched to 100 nt and the coverage values adjusted. Heatmaps were generated using a custom MATLAB (MATLAB and Statistics Toolbox Release 2016a The MathWorks, Inc., Natick, Mass., United States) script where a rolling average of 25 nt was employed.

sRNA-Seq and PAR-CLIP

A bowtie index was built using bowtie-build (14) for tRNA gene sequences extended by 7 nt on each side. MicroRNA and snoRNA sequences were downloaded from UCSC (13) and an index was built the same way. sRNA-seq data for Dicer knockdown and scrambled shRNA control (3 reps) and PAR-CLIP data for AGO 1, 2, 3 and 4 (15) and Dicer (16) (3 reps) were adapter trimmed using cutadapt 1.8.3 with --minimum-length 10²⁴. Further sequences consisting of partial adapters were removed using a custom written perl script. The remaining sequences were mapped to tRNAs±7 nt/mi- & snoRNAs using bowtie -S -v3 --all --best -strata (14). Only sequences between 19 nt and 22 nt in length were considered for further analysis. Reads were equalized to their genomic sequence.

For sRNA analyses, reads supported by at least one AGO and one Dicer PAR-CLIP hit in either rep. Hits in tRNA/miRNA/snoRNA regions were normalized to total number of mappable reads to the genome. These were determined by mapping reads with bowtie (14) -m 1 -k 1 to hg38 and adding up “reads with at least one reported alignment” and “reads with alignments suppressed due to -m” from the output report. For PAR-CLIP reads occurring 25 times in all AGO sets and 323, 41 or 19 times in either Dicer rep1, rep2 or rep3 sets respectively were considered further (cut-offs were due to different library sizes and distribution of read occurrence). These were considered to be tsRNAs.

ChrRNA-Seq

cDNA and ncRNA sequence data was downloaded from Ensembl version 89 (17) and a kallisto (v0.43.1) index was built. Read counts for RNA-seq data were generated using kallisto (18) with the following options: --rf-stranded -b 100 -t5.

Differential Gene Expression

Differentially expressed genes were determined with DESeq2 (19). For mRNA-seq genes with the FDR adjusted P<0.001 were considered as significantly differentially expressed. For chrRNA-seq, genes with the adjusted P<0.005 for shDicer and shAgo2 were considered as significantly differentially expressed. Due to the low number of changing genes, a less stringent criterion of adjusted P<0.05 was used in shDrosha samples.

Target Prediction

tsRNA targets were predicted by running miRanda 3.3a (20) with the parameters -sc 150 -en -30 -quiet against significantly upregulated genes determined from mRNA-Seq. The same analysis was repeated for genes significantly upregulated in shDicer and shAgo2 in chrRNA-seq.

tsRNA Distribution

sRNAs mapping to tRNAs were grouped if they overlapped each other by 10 nt. Each group was then considered as one sRNA with the most extreme mapping. Each tRNA was stretched to 100 nt. The absolute frequency was computed as number of (grouped) sRNA hits in each tRNA position.

Disease-Association Heatmap

The inventor downloaded the table for all gene-disease associations as well as a curated annotation of cui, disease and disease classes from DisGeNET (21). DisGeNET uses NCBI annotation as reference. We extracted the gene symbols and all synonyms for NCBI genes. The inventor also extracted gene symbols for Ensembl 89 using BioMart (22). The gene universe was taken as the overlap of NCBI gene symbols and Ensembl 89 gene symbols and only disease and target genes with symbols in this universe were considered further. A contingency table was thus constructed, and a one-sided Fisher exact test was employed to assess the significance of the observation. Heatmaps were plotted in R (23) using the pheatmap package (24) by constructing a binary matrix for target-gene—disease/disease class associations. This matrix was ordered first by column sums (genes), then by row sums (diseases).

tRNA Secondary Structure Prediction

To predict possible tRNA structures we used the mFold (25) web server with % suboptimality: 20 and otherwise default parameters.

### DISCUSSION

The advent of deep sequencing techniques has helped to identify tsRNAs in mammalian cells, dispelling the suspicion that the sRNAs originating from tRNAs are random fragmentation products. However, it remains controversial as to which enzymes generate tsRNAs and the extent of their biological role has remained unclear. As Dicer has been implicated in producing some tsRNAs the present inventor sought to explore if the nuclear function of Dicer has any relation to this class of sRNAs.

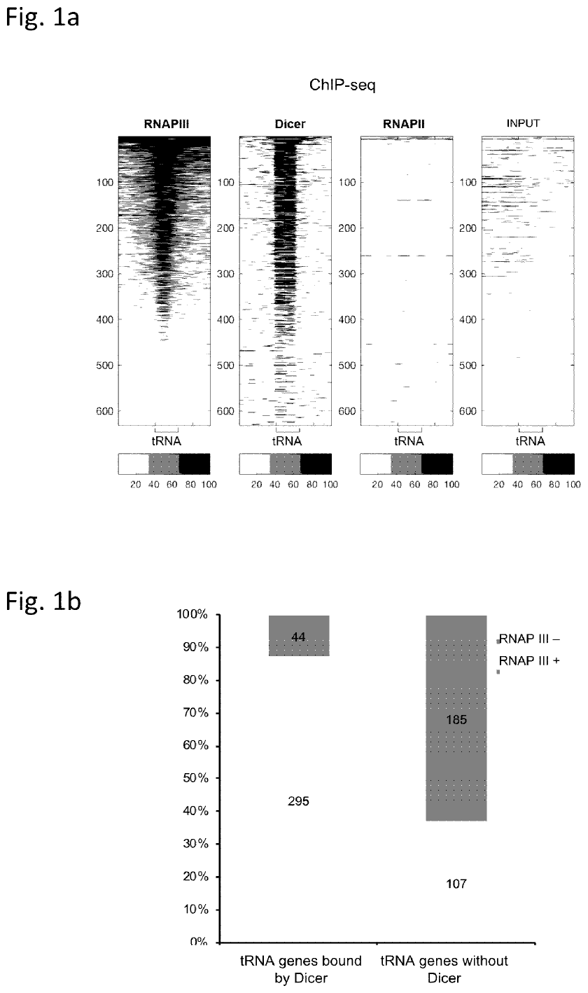

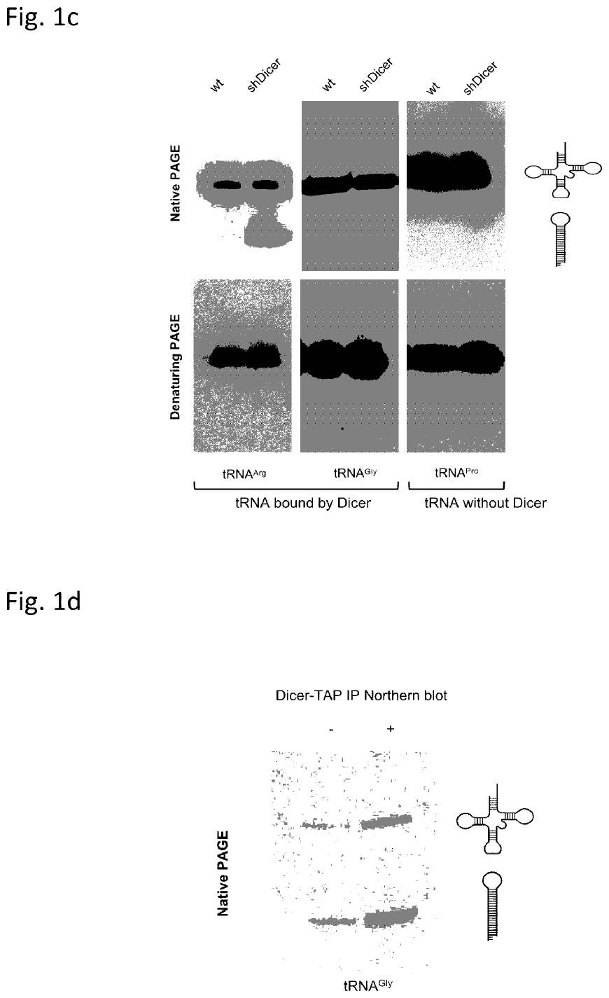

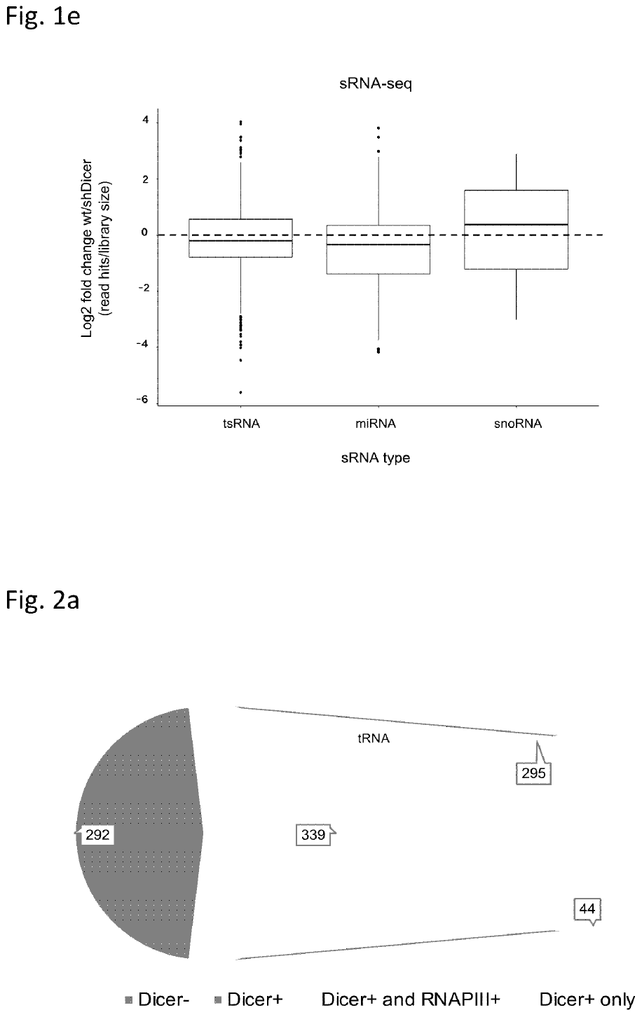

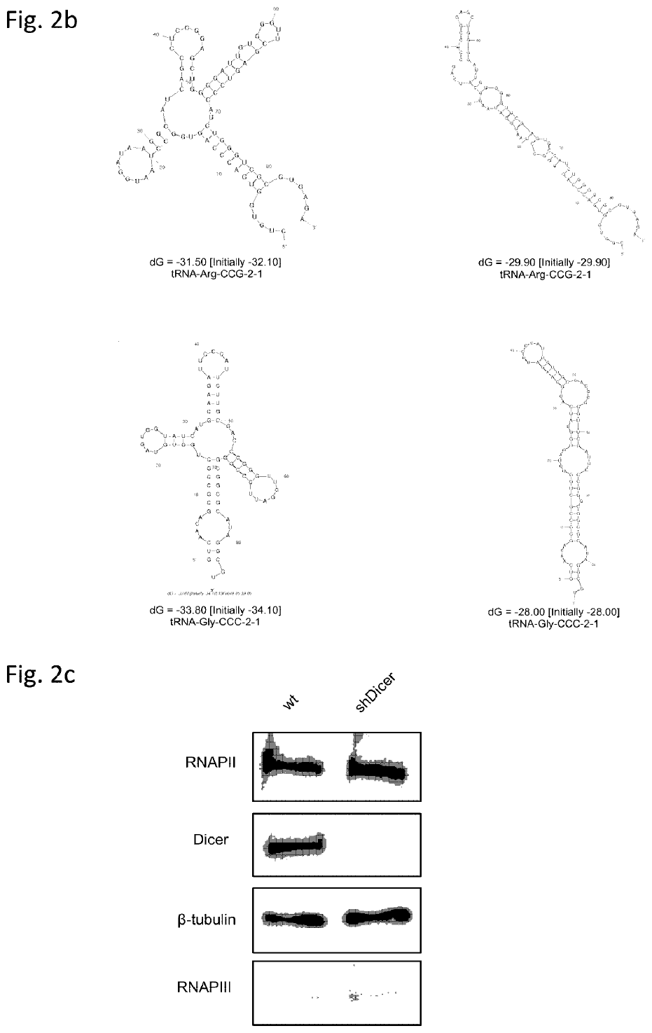

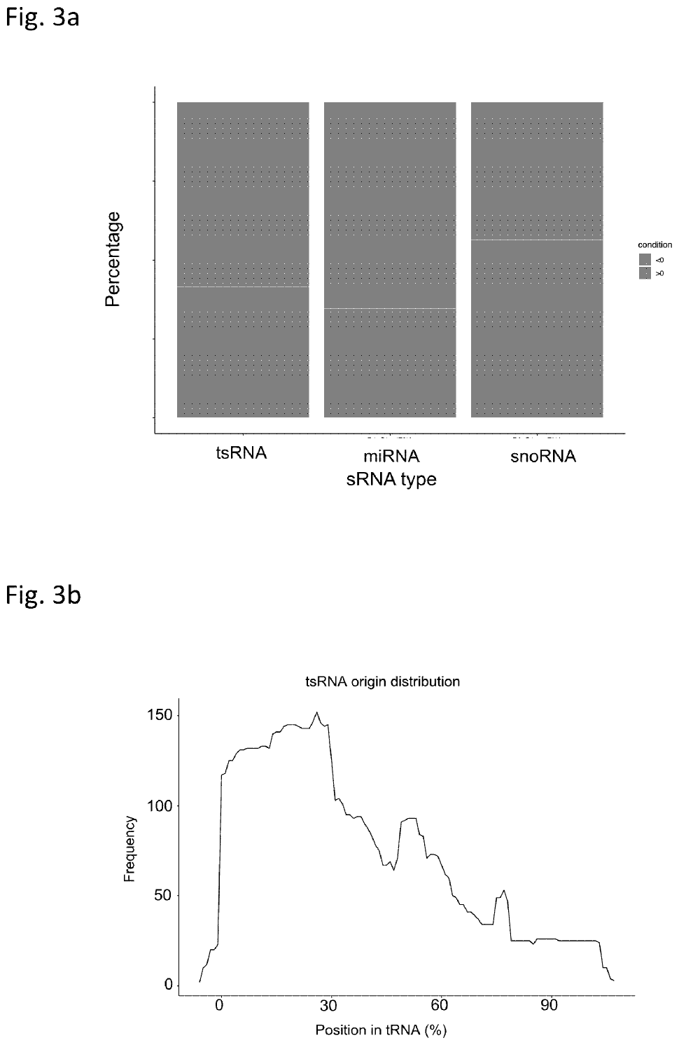

The inventor first employed Dicer and RNA polymerase III (RNAPIII) chromatin immunoprecipitation sequencing (ChIP-seq) data to show that Dicer preferentially binds to transcribed tRNA genes. RNA polymerase II (RNAPII) and input were used as negative controls. (FIG. 1a, b, FIG. 2a). To test whether Dicer has any effect on tRNA, the inventor performed Northern blot analyses and showed that upon Dicer knockdown a distinct population of tRNAs was stabilised in native polyacrylamide gel electrophoresis (PAGE) but not in denaturing PAGE (FIG. 1c), suggesting that this population of tRNAs has an identical primary sequence as canonical tRNAs, but is folded into an alternative secondary structure. The inventor also used mFold (25) to predict alternative secondary structures of tRNAs and showed that indeed they can fold into short hairpin structures, resembling miRNA precursors (FIG. 2b). To confirm the direct association between Dicer and the alternatively folded tRNAs, the inventor immunoprecipitated tandem affinity purification (TAP)-tagged Dicer and detected enrichment of alternatively folded tRNAs in native PAGE (FIG. 1d). It has been proposed that Dicer binds various RNA substrates, but without any further processing. To test whether Dicer is processing alternatively folded tRNAs into functional sRNAs, the inventor sequenced and compared sRNAs isolated from wild type and inducible Dicer knockdown cells (FIG. 2c). First, the inventor detected tsRNA in wildtype cells, confirming their existence. Secondly, their levels were decreased upon Dicer knockdown, along with miRNAs, which the inventor used as positive controls. Levels of small nucleolar RNAs (snoRNAs), used as negative controls, did not decrease upon Dicer knockdown (FIG. 1e, FIG. 3a). A large proportion of these tsRNAs derived from the first half of tRNAs (FIG. 3b), which distinguishes them from tRNA fragments. These data suggest that Dicer is involved in the biogenesis of tsRNAs derived from alternatively folded tRNA structures.

Therefore, this type of tsRNA is different from previously reported Dicer-independent tsRNA derived from mature tRNA.

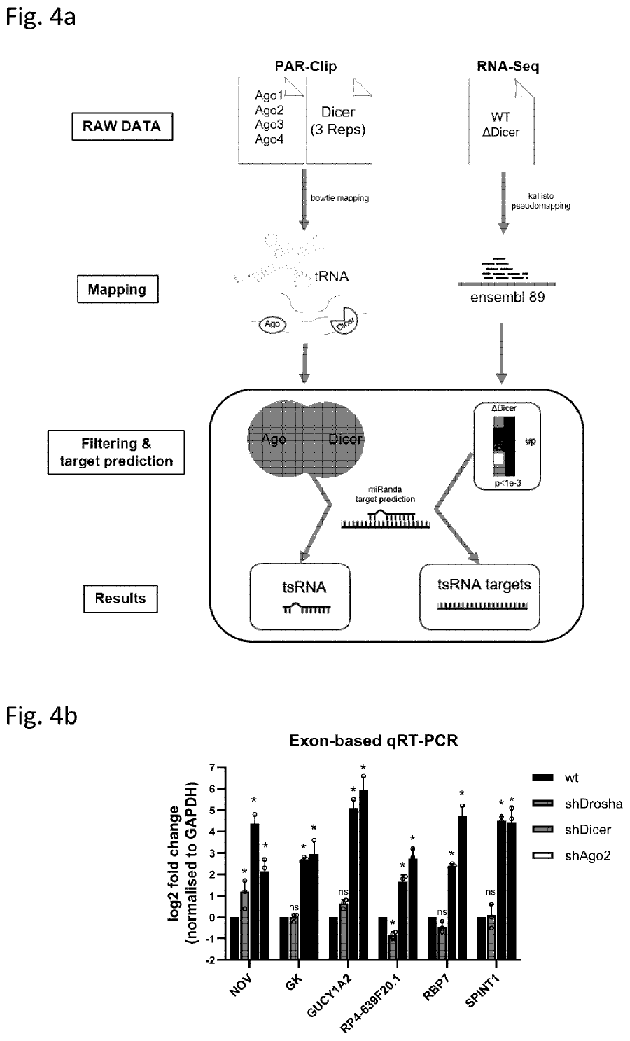

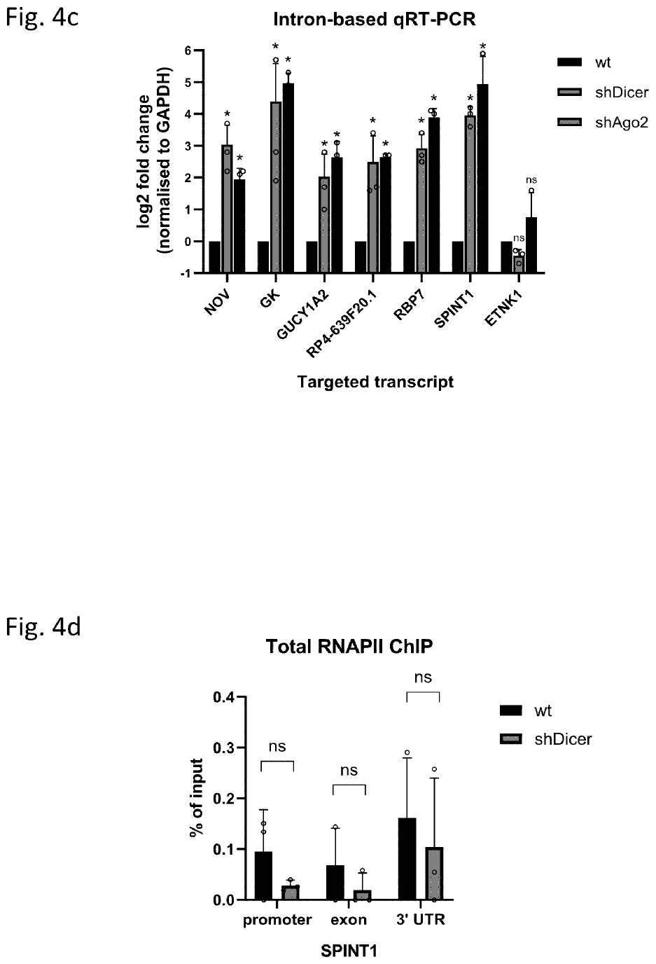

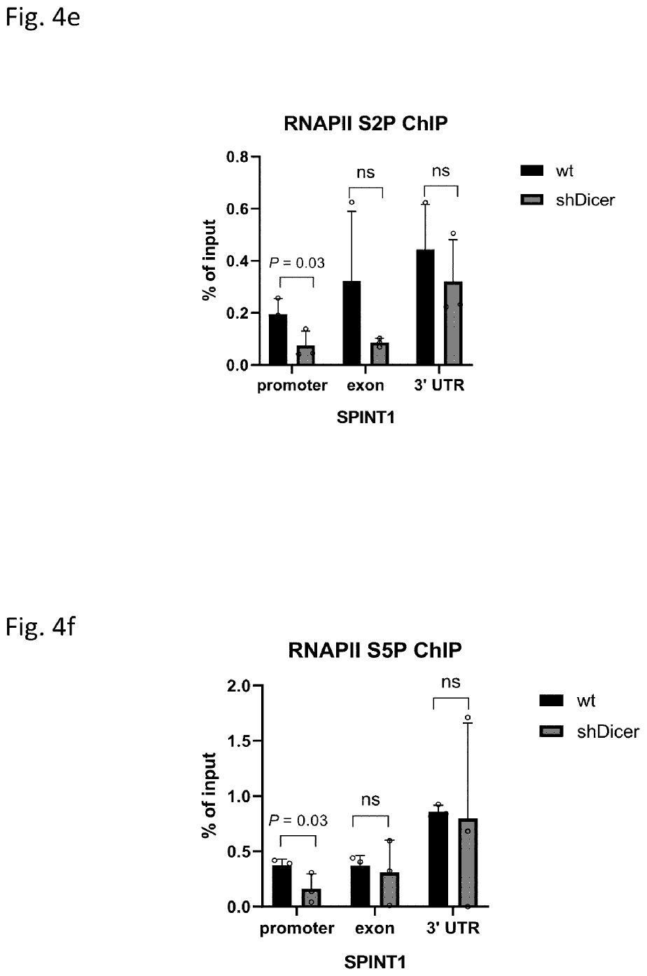

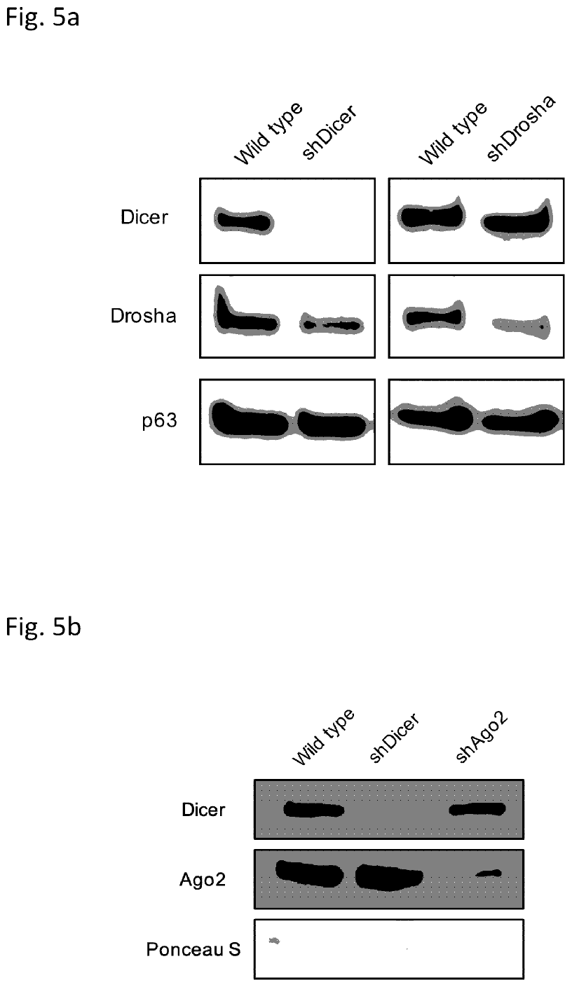

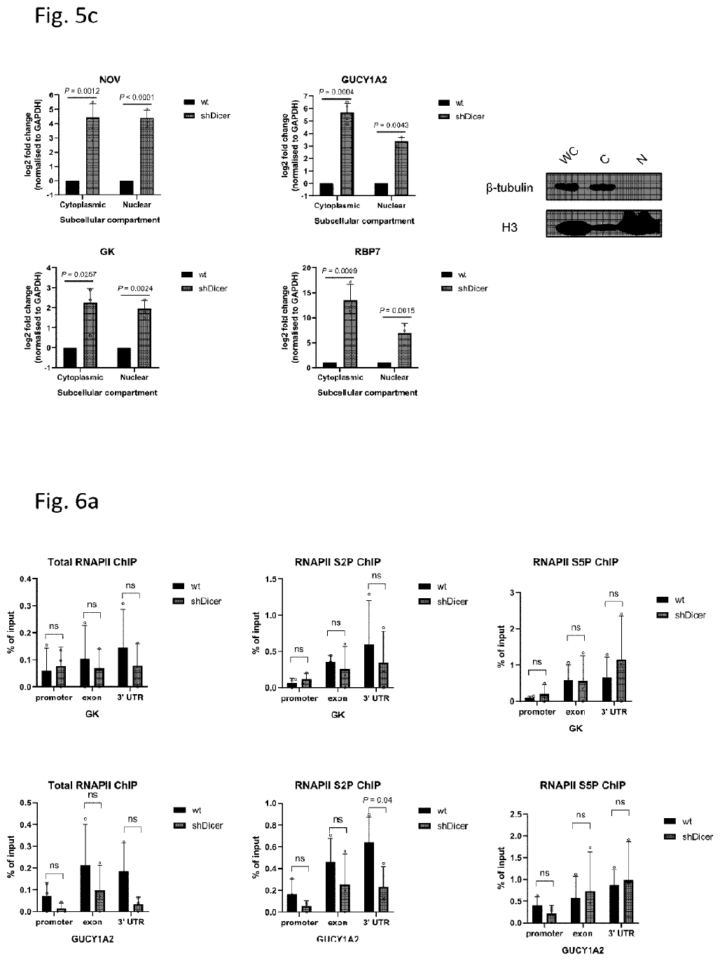

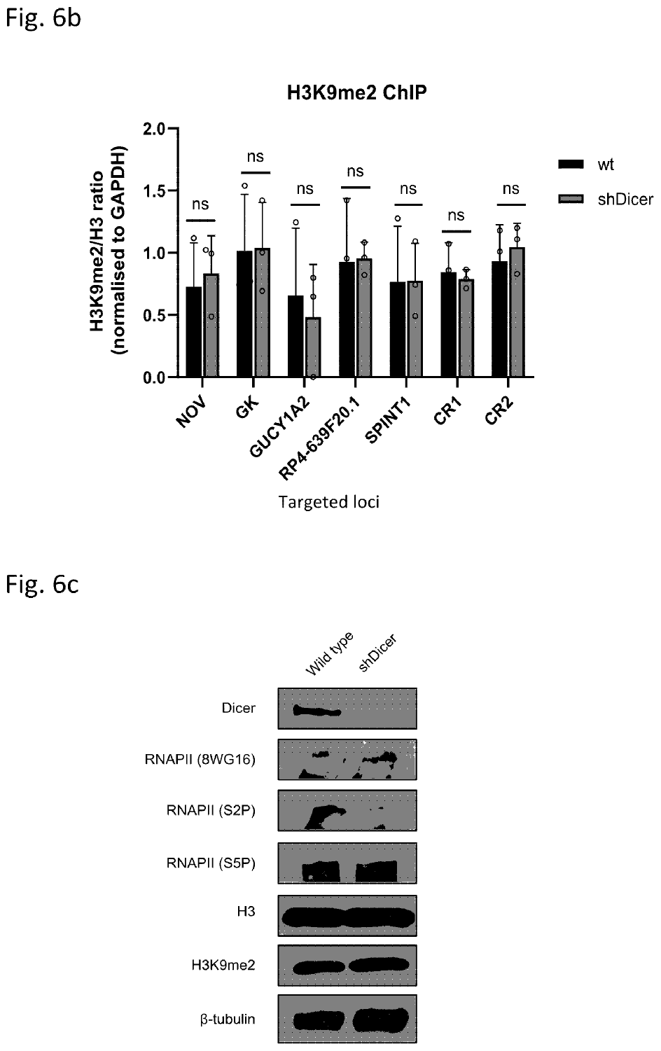

The inventor next extracted sequences from photoactivable ribonucleoside-enhanced crosslinking and immunoprecipitation (PAR-CLIP) Dicer and Ago1, 2, 3 and 4 data and mapped these to tRNA genes (±7 nt). Next, the inventor sequenced mRNA from wildtype and Dicer knockdown cells and determined genes upregulated in Dicer knockdown via DESeq2. Finally, they employed miRanda (20) to generate a list of genes that are predicted to be targeted by Dicer- and Ago-associated tsRNAs (FIG. 4a). Six randomly selected target genes were tested by reverse transcription-quantitative PCR (qRT-PCR) in wild type, Drosha, Dicer and Ago2 knockdown cells (FIG. 5a, b) using primers targeting the 3′ untranslated region (UTR). If the genes are regulated by miRNAs, absence of Drosha should result in their upregulation as Drosha is crucial for miRNA biogenesis. Four protein-coding genes (GK, GUCY1A2, RBP7 and SPINT1) and a non-coding transcript (RP4-639F20.1) were upregulated only in Dicer and Ago2 knockdown, indicating that they are not regulated via a miRNA-dependent pathway (FIG. 4b). NOV was significantly upregulated upon knockdown of Drosha, Dicer and Ago2. As these data suggest miRNA-independent regulation for most of the tested genes, we analysed next in which subcellular compartments the target genes were upregulated and performed subcellular fractionation followed by qRT-PCR. Surprisingly, four tested target genes, were upregulated in both cytoplasmic and nuclear compartments (FIG. 5c). One could argue that potential contamination from the cytoplasmic fraction could lead to this observation. Therefore, the inventor used qRT-PCR probes in the introns of the target genes and showed that six tested genes were upregulated upon Dicer and Ago2 knockdown while levels of non-target control gene ETNK1, were not affected (FIG. 4c). These results imply that the tested genes are regulated at the transcriptional level (rather than post-transcriptionally) as splicing of introns happens co-transcriptionally. Transcriptional gene silencing (TGS) is mediated through histone modifications and heterochromatin formation and results in transcriptional shut down and absence of RNAPII on the chromatin. To test whether tsRNA can target genes for TGS, the inventor performed ChIP of total and active forms of RNAPII (phosphorylated at serine positions 2 (S2P) and 5 (S5P) in the C-terminal domain) at three selected target genes (probing in promoter, exon and 3′UTR) and found that levels of RNAPII do not increase upon Dicer knockdown (FIG. 4d, FIG. 6a). Furthermore, the levels of di-methylated histone 3 at lysine 9 (H3K9me2), which is a heterochromatin mark, were detected only at the background level at the target genes and did not change upon Dicer knockdown, while overall protein levels of RNAPII, H3 and H3K9me2 were not affected (FIG. 6b, c). These results suggest that tsRNA-mediated gene silencing does not require changes in transcription or chromatin state, but rather leads to nascent RNA degradation.

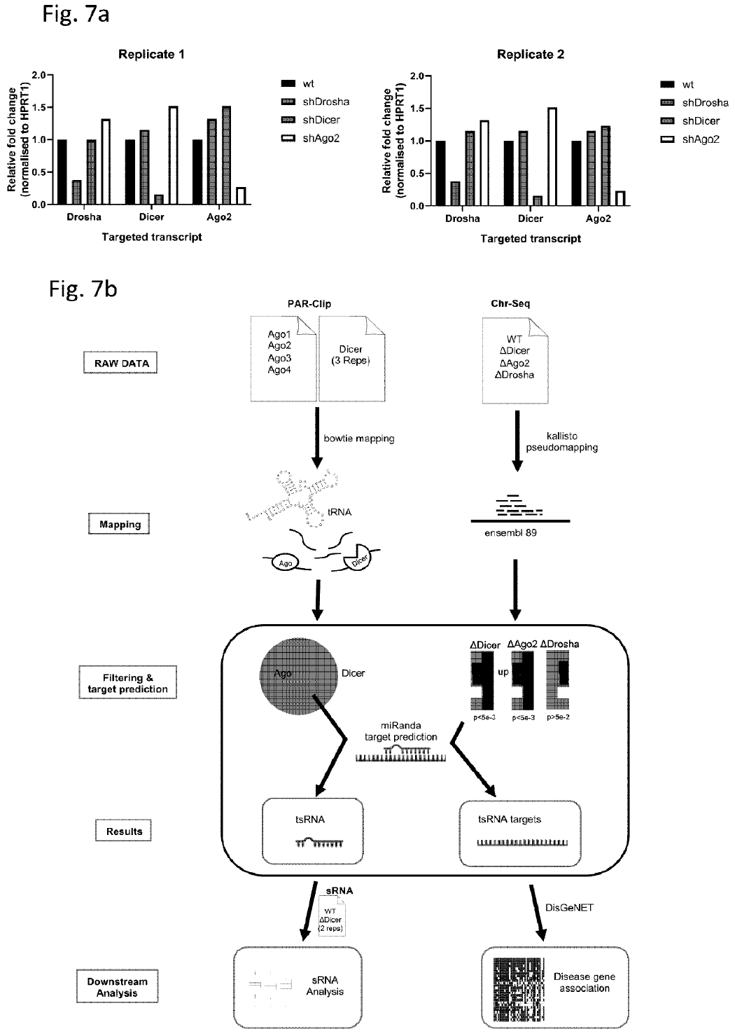

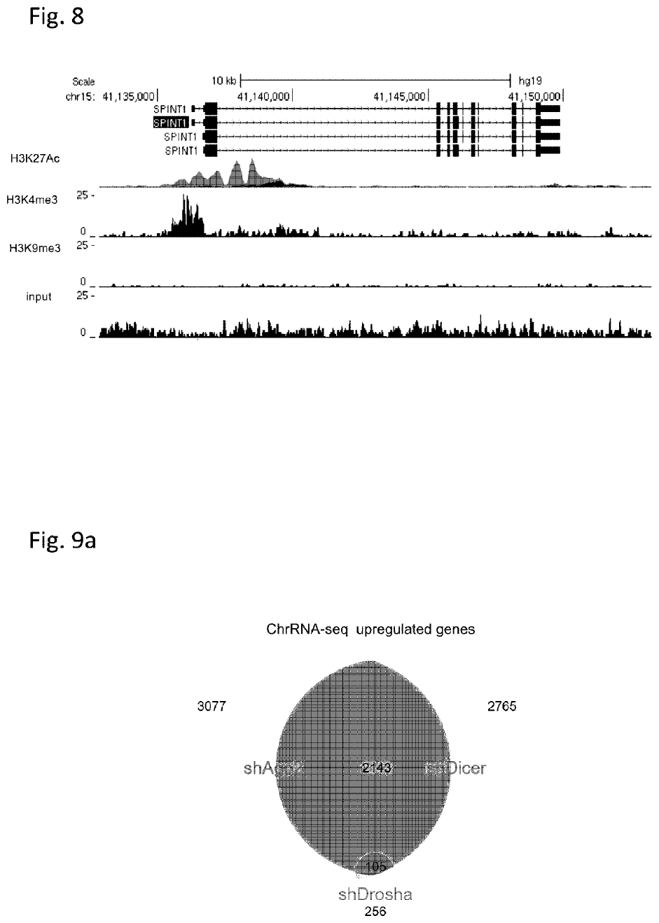

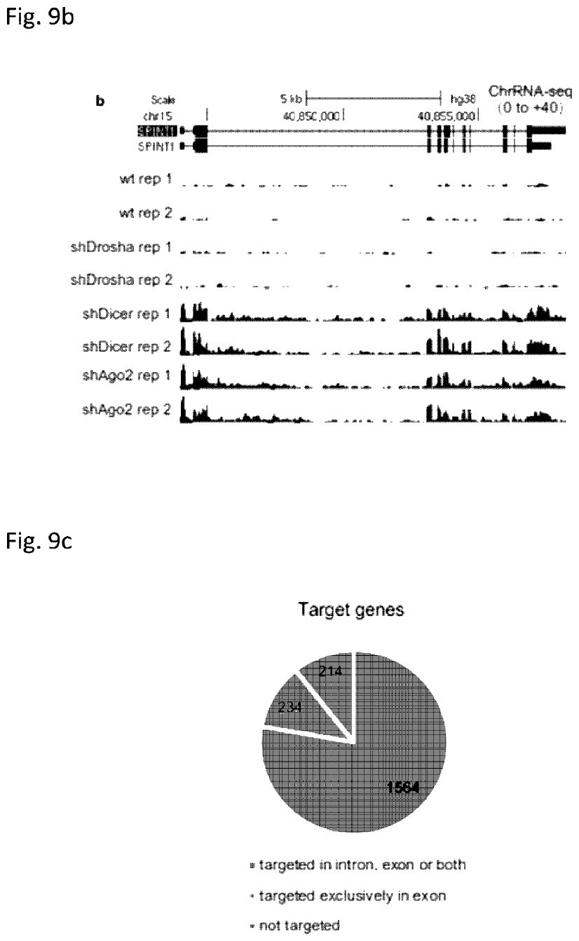

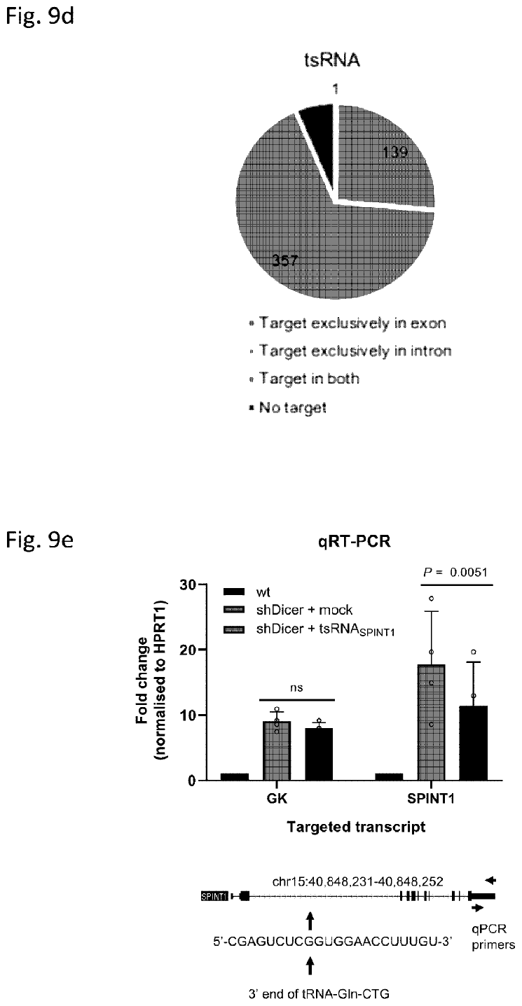

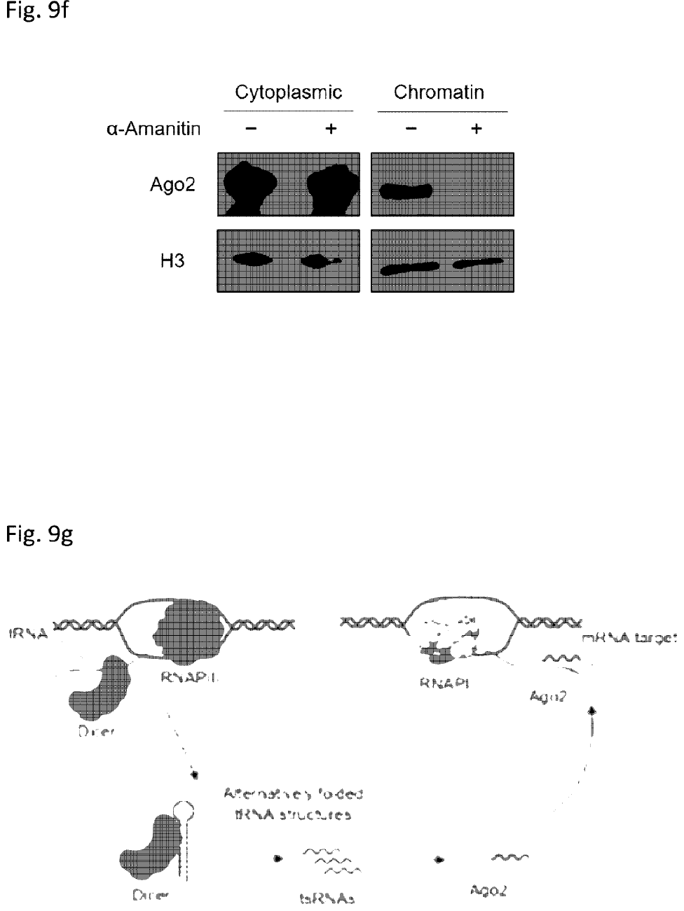

To identify genes that are regulated globally by this distinct gene silencing mechanism, the inventor performed chromatin-associated RNA sequencing (chrRNA-seq) to detect levels of nascent transcripts in wild type, Drosha, Dicer and Ago2 knockdown cells (FIG. 7a). Over 2000 genes that were upregulated upon both Dicer and Ago2 knockdown, but not upon Drosha knockdown (FIG. 9a, b) were identified. Close inspection of one of the target genes, SPINT1, confirmed only low levels of nascent RNA in wild type and Drosha knockdown cells. However, active histone marks H3 acetylated at lysine 27 (H327Ac) and H3 trimethylated at lysine 4 (H3K4me3) are present at the promoter of SPINT1 while repressive mark trimethylated H3 at lysine 9 (H3K9me3) was not detected (FIG. 8; GEO accession number GSE66530). The inventor subsequently performed miRanda analysis on the target genes upregulated in the chrRNA-seq (FIG. 7b) and show that they are targeted by tsRNAs and specifically in their introns (FIG. 9c). Of the 531 tRNAs that produce tsRNAs, 496 have targets in at least one intronic region of protein coding genes (FIG. 9d). To verify the molecular mechanism of tsRNA targeting introns, the inventor synthesized tsRNA sequence predicted to target the second intron of SPINT1, transfected wild type and Dicer knockdown cells and assess the levels of SPINT1 and GK mRNA using qRT-PCR. The inventor found that the upregulation of SPINT1 upon Dicer knockdown was significantly reduced after transfection with its targeting tsRNA, while the upregulation of GK, used as a negative control, was not affected (FIG. 9e). This experiment demonstrates that tsRNA can target the intronic region of a specific gene to downregulate its expression. As the target genes are upregulated upon Dicer and Ago2 knockdown and Ago2 normally functions downstream of Dicer, the inventor hypothesised that Ago2 is guided by the tsRNA to the chromatin to target nascent RNA for degradation. The inventor observed that levels of Ago2 on the chromatin decreased upon inhibition of RNAPII by a-Amanitin for 24 hours (FIG. 9e), suggesting that the association between Ago2 and the chromatin is transcription-dependent. Altogether the inventor propose a novel gene silencing mechanism which employs Dicer-dependent tsRNA to drive Ago2-dependent nascent RNA degradation (FIG. 9g). This mechanism is distinct from miRNA-mediated posttranscriptional gene silencing as it takes place in the nucleus and is Drosha-independent. It also differs from transcriptional gene silencing as it does not involve transcriptional inhibition and heterochromatin formation. An advantage of this gene silencing mechanism is that it does not require altering the chromatin context, which can potentially affect the expression of genes that are in the vicinity of target genes.

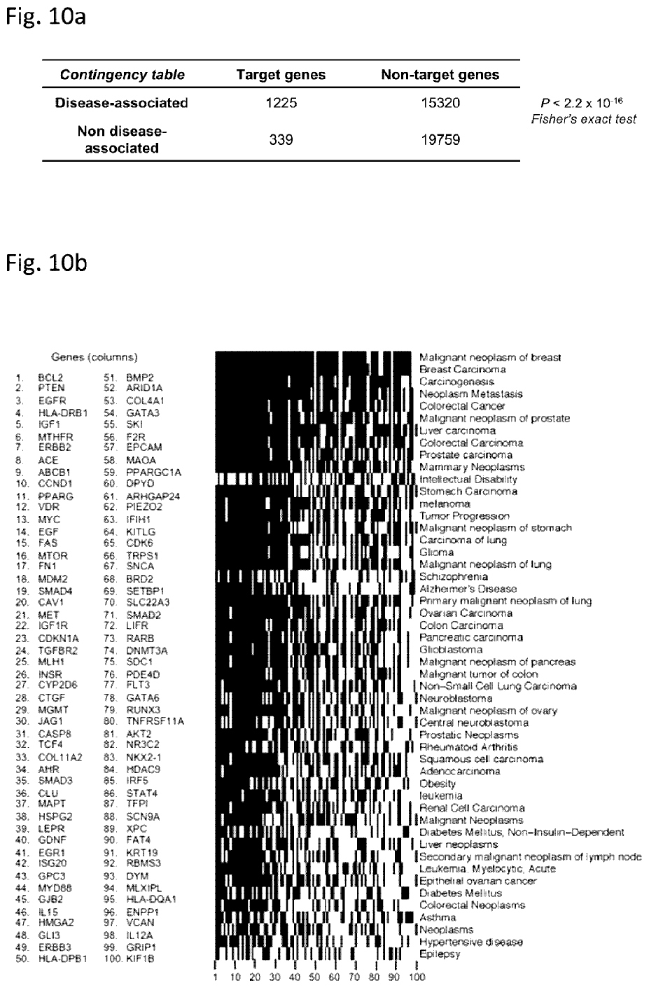

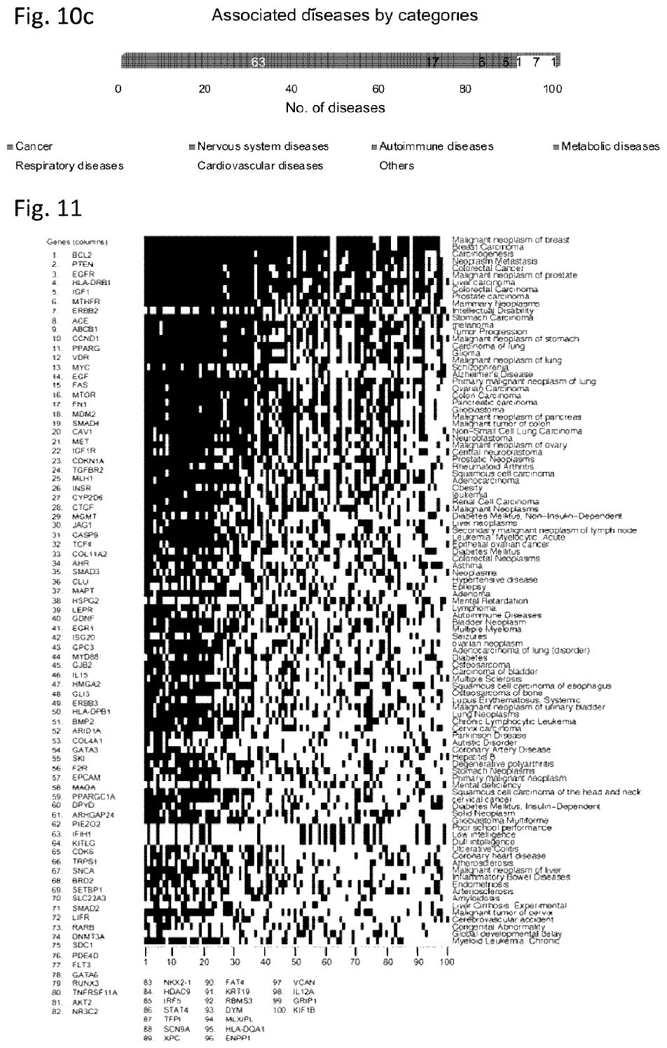

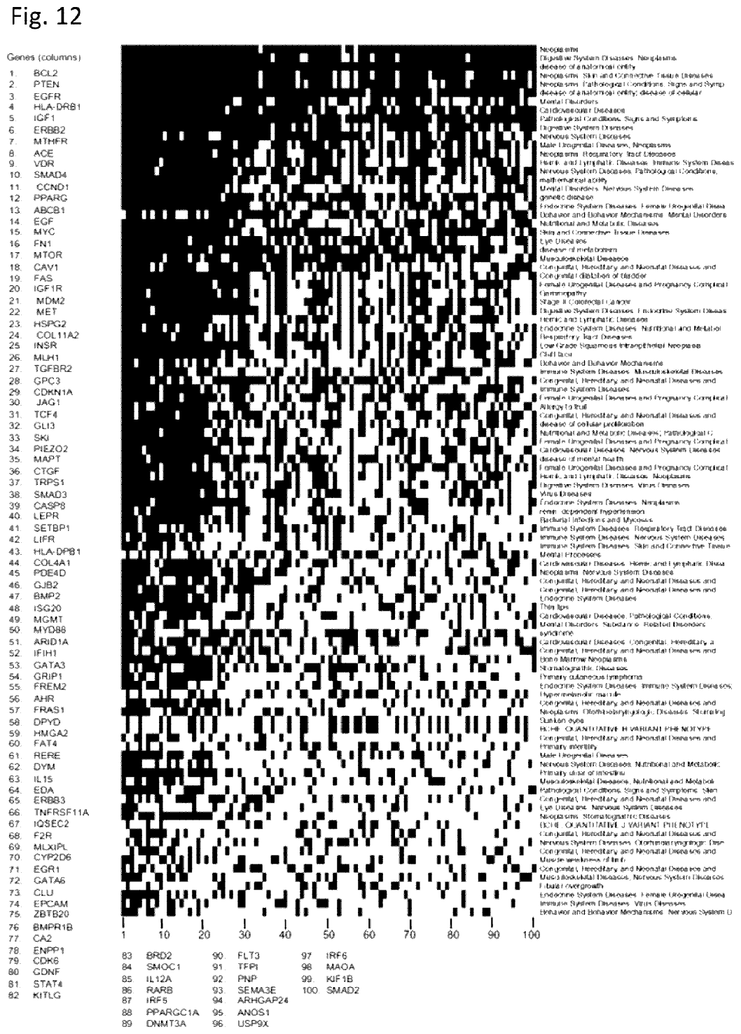

Finally, the inventor investigated what the tsRNA regulated genes have in common. Considering that they are suppressed in wildtype cells, we asked whether their nascent RNA degradation has a biological context. The inventor employed DisGeNET, a platform documenting human disease-related genes and surprisingly found that the genes targeted by Dicer-dependent tsRNAs for silencing are significantly disease-associated, when compared to non-target genes (one-sided Fisher's exact test, P<2.2×10-16) (FIG. 10a). The inventor identified association with at least one disease for 1225 target genes (of 1564 total) (FIGS. 11 and 12). In FIG. 10b the inventor show the top 100 genes involved in the largest number of diseases (here top 50) as known to date. Furthermore, the inventor categorised the top 100 diseases that are associated with the target genes and uncovered that many target genes are involved in oncogenesis, nervous system diseases, autoimmune diseases and more (FIG. 10c).

The inventor of the present invention proposes a distinct mechanism of gene silencing. Unlike miRNA-mediated PTGS, Dicer-dependent tsRNAs target genes in nucleus co-transcriptionally. However, unlike TGS facilitated through transcriptional repression and heterochromatin formation, these tsRNAs target introns of protein coding genes for immediate nascent RNA degradation. This novel molecular mechanism regulating 1125 disease-associated genes has a great translational potential in the current era of expanding RNA therapeutics.

### Example 2: EGFR Expressing Cell Line

The tsRNA was generated as shown in FIG. 4a. Gene encoding epidermal growth factor receptor; EGFR is a member of the type I family of growth factor receptors whose gene is located on chromosome 7p12 and encodes a 170 kDa transmembrane glycoprotein with tyrosine kinase activity. The high level expression of EGFR in many cancerous sites has been repeatedly correlated with more malignant or advanced disease, poor prognosis.

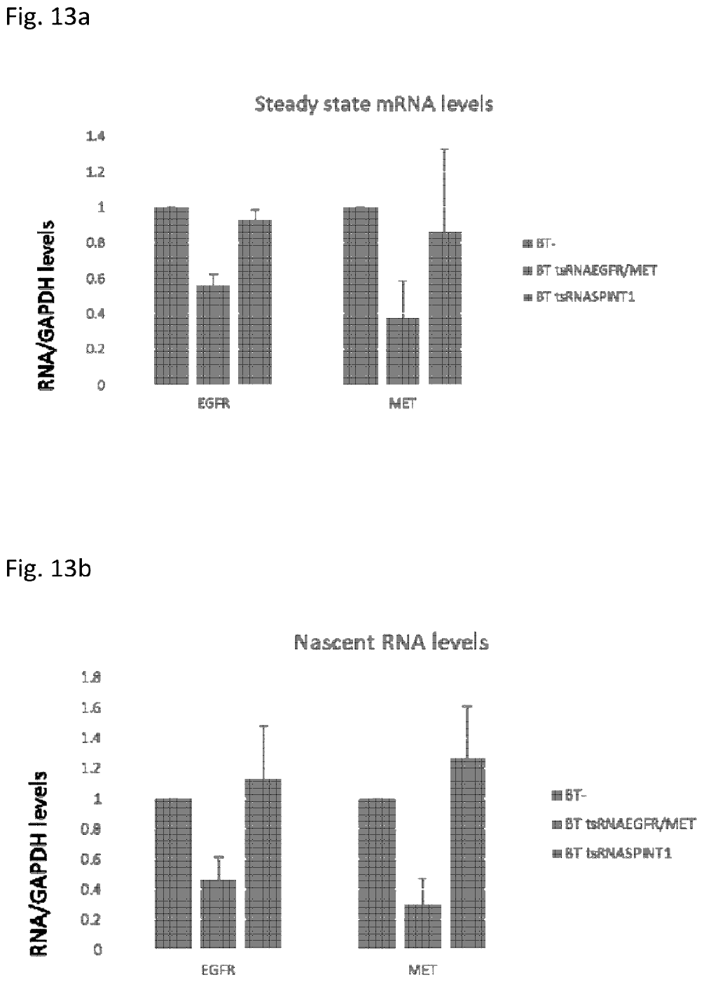

Cells were transfected with tsRNAs for 24 hours and collected for RNA isolation. Relative RNA levels were quantified by reverse transcription-quantitative PCR (qRT-PCR). Primers targeting exons were used for quantifying steady state mRNA levels, while primers targeting introns were used quantifying nascent (newly produced) RNA levels. As shown in FIG. 13A, steady state levels of EGFR mRNA were reduced upon transfection of tsRNA EGFR. As shown in FIG. 13B, nascent levels of EGFR mRNA were reduced upon transfection of tsRNA EGFR.

### Example 3: MET Expressing Cell Line

The tsRNA was generated as shown in FIG. 4a. cMET also is overexpressed in breast cancer cells and human breast tumours and its expression correlates with EGFR expression. cMET growth factor receptor is characterized as a receptor tyrosine kinase. cMET, in part, regulates EGFR tyrosine phosphorylation and growth. Cells were transfected with tsRNAs for 24 hours and collected for RNA isolation. Relative RNA levels were quantified by reverse transcription-quantitative PCR (qRT-PCR). Primers targeting exons were used for quantifying steady state mRNA levels, while primers targeting introns were used quantifying nascent (newly produced) RNA levels. As shown in FIG. 13A steady state levels of MET mRNA were reduced upon transfection of tsRNA MET. As shown in FIG. 13B nascent levels of MET mRNA were reduced upon transfection of tsRNA MET.

### Example 4: EGFR+MET Using BT549 which Expresses Both

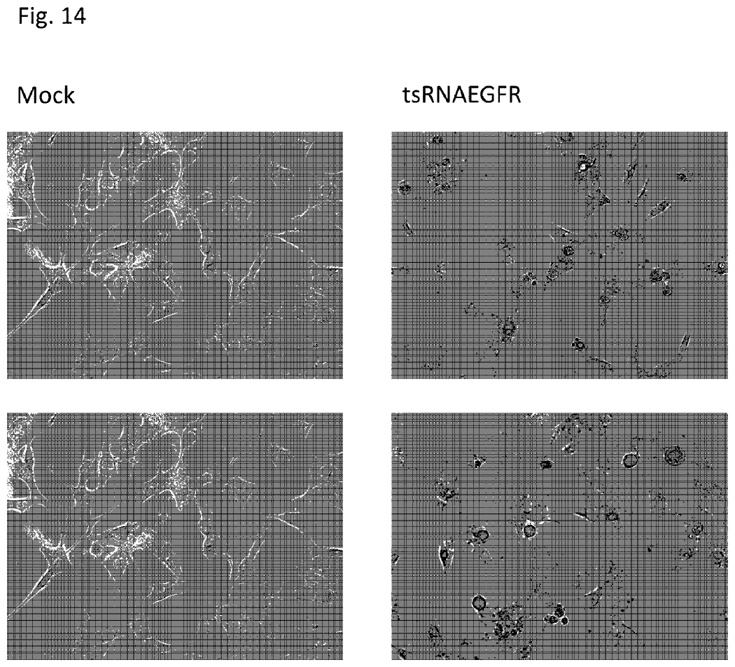

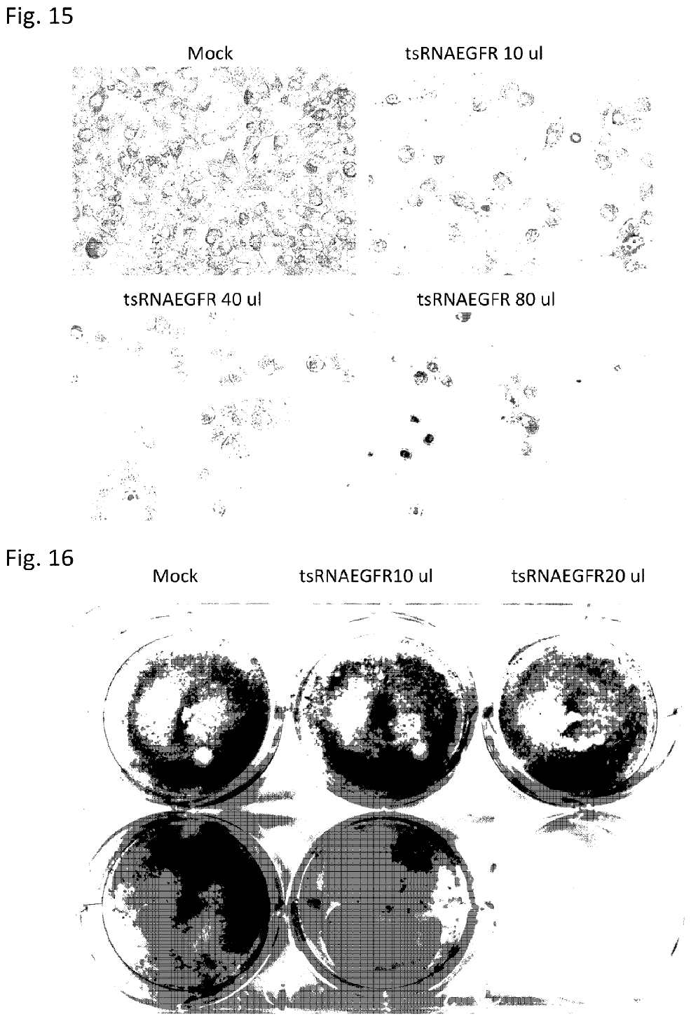

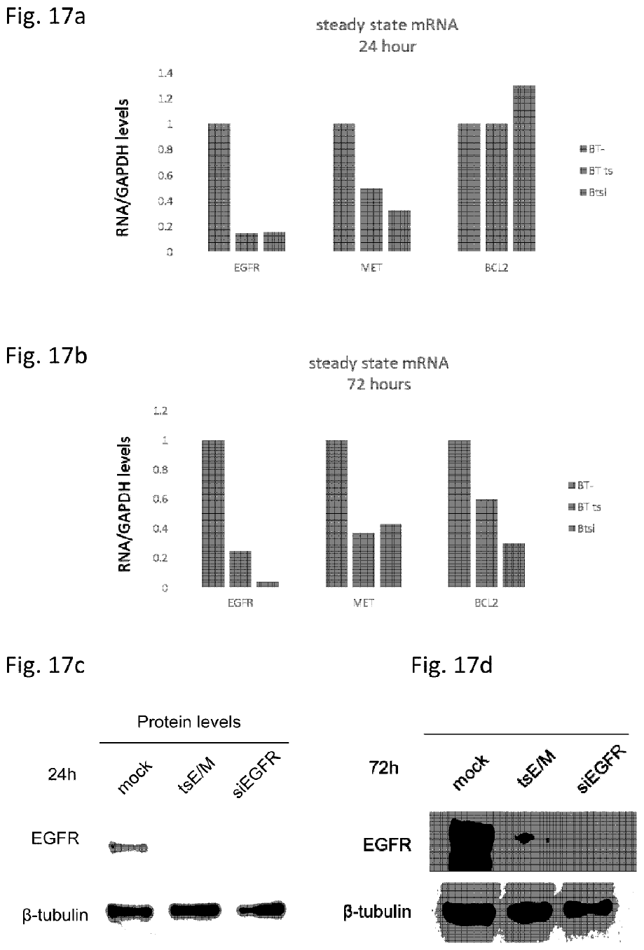

As shown in FIG. 14, BT549 Cells were transfected with tsRNA EGFR/MET and imaged on day 3 using a light microscope. More cells appeared to be dead in the population of cells transfected with tsRNA EGFR/MET. The tsRNA is single stranded and has the following sequence (5′ to 3′): UCCCUGGUGGUCUAGUGGUUAG (SEQ ID NO. 4). BT549 cells were transfected with increasing concentration of tsRNA EGFR/MET (10, 20, 40 and 80 ul—100, 200, 400 and 800 pmol) and imaged on Day 6. As shown in FIG. 15, the number of dead cells increased with increasing amount of tsRNA EGFR/MET (light microscopy) and in FIG. 16, it can be seen that the number of live cells decreased with increasing amount of tsRNA EGFR/MET (crystal violet staining). BT549 cells transfected either with tsRNA EGFR/MET (100 pmol) or siRNA targeting EGFR (100 pmol) for 24 h. Total RNA and protein were extracted from the cells for qRT-PCR (FIGS. 17A and 17B) and western blot (FIGS. 17C and 17D) respectively.

### Example 5: BCL2

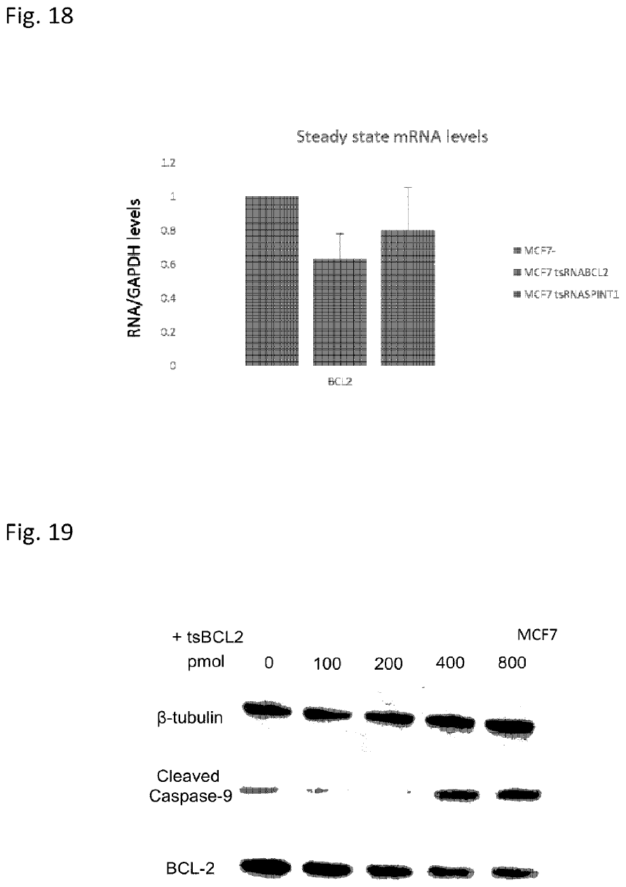

The tsRNA was generated as shown in FIG. 4a. It is single stranded and has the following sequence (5′ to 3′): UAAGCCAGGGAUUGUGGGUUCG (SEQ ID NO. 5). Bcl-2 protein family plays a key role in regulation of apoptosis including necrosis and autophagy. The overexpression of antiapoptotic gene of the Bcl-2 family namely Bcl-2 is responsible for resistance to breast cancer chemotherapy. MCF7 is a breast cancer cell line expressing BCL2. tsRNA BCL2 used here is targeting BCL2 specifically, tsRNA SPINT1 was used as a control. Cells were transfected with tsRNAs for 24 hours and collected for RNA isolation. Relative RNA levels were quantified by reverse transcription-quantitative PCR (qRT-PCR). Primers targeting exons were used for quantifying steady state mRNA levels. Transfection of tsRNA BCL2 into cells led to downregulation of steady state BCL2 mRNA levels (FIG. 18).

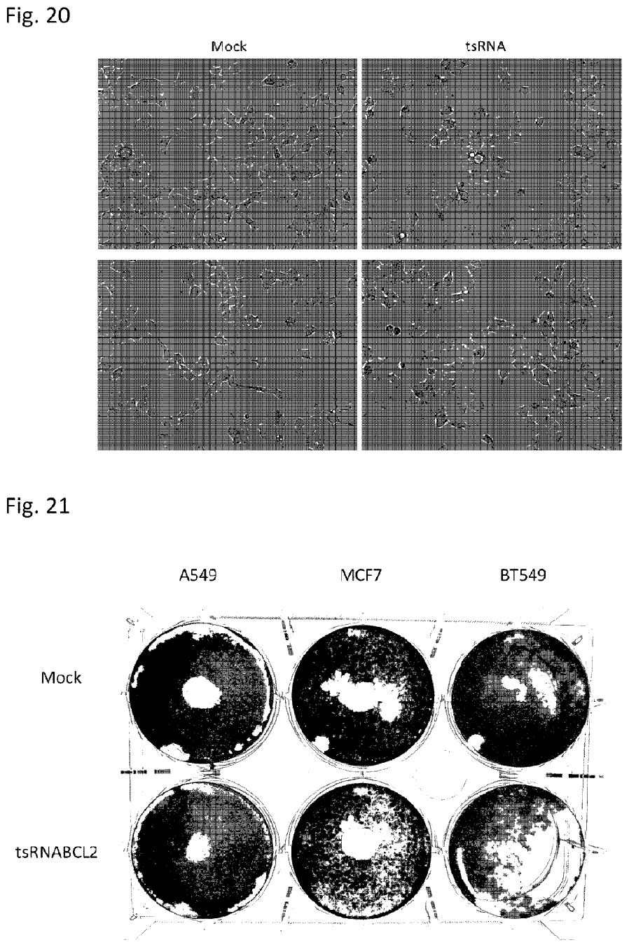

MCF7 cells were transfected with increasing amount of tsRNA BCL2 for 24 hours. Total protein was extracted for western blot and beta-tubulin signals were used as loading control. Cleaved Caspase-9 signals were used as proxy for cells undergoing apoptosis. As shown in FIG. 19, BCL-2 levels decreased with increasing amount of tsRNA BCL2 transfected. Cleaved Caspase-9 followed the opposite pattern—increased with increasing amount of tsRNA BCL2, indicating more cells were undergoing apoptosis. MCF7 cells were transfected with tsRNA BCL2 and imaged on day 3 with light microscopy. More dead cells were present in population of cells transfected with tsRNA BCL2. This is shown in FIG. 20.

A549, MCF7 and BT549 cells were transfected with tsRNA BCL2 and were subjected to crystal violet staining on day 8. As is shown in FIG. 21, fewer live cells were present in populations of cell transfected with tsRNA BCL2.

### Example 6: LINC00665

The tsRNA was generated as shown in FIG. 4a. It is single stranded and has the following sequence (5′ to 3′): GGGGGUGUAGCUCAGUGGUA (SEQ ID NO. 6). Long non-coding RNAs (IncRNAs) are frequently dysregulated in multiple malignancies, demonstrating their potential oncogenic or tumour-suppressive roles in tumorigenesis. LINC00665 is markedly upregulated in lung cancer tissues and might serve as an independent predictor for poor prognosis. Functional assays indicated that LINC00665 reinforced lung cancer cell proliferation and metastasis in vitro and in vivo. LINC00665 regulates pathways in the cell cycle to facilitate the development and progression of cancer through ten identified core genes: CDK1, BUB1B, BUB1, PLK1, CCNB2, CCNB1, CDC20, ESPL1, MAD2L1, and CCNA2. A549 is an invasive lung cancer cell line which expressing LIN000665. tsRNA LIN000665 used here is targeting LIN000665, tsRNA SPINT1 was used as a control.

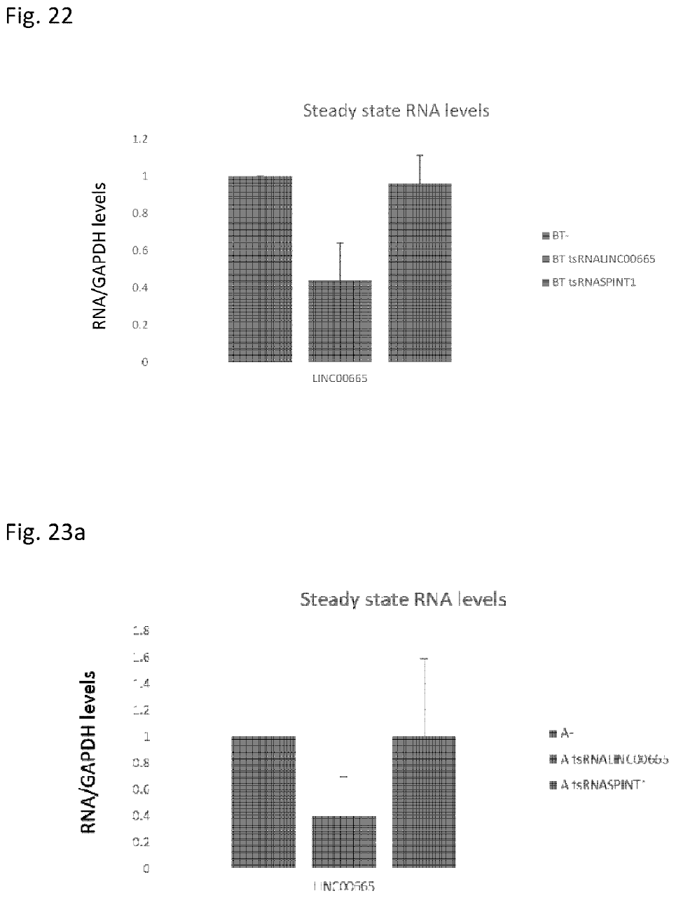

BT549 cells were transfected with tsRNA LIN000665 for 24 hours. RNA was extracted for qRT-PCR. Primers targeting the exons were used for quantifying steady state RNA levels. As shown in FIG. 22, LINC0665 levels were reduced by transfection of tsRNA LINC0665 in BT549 cells.

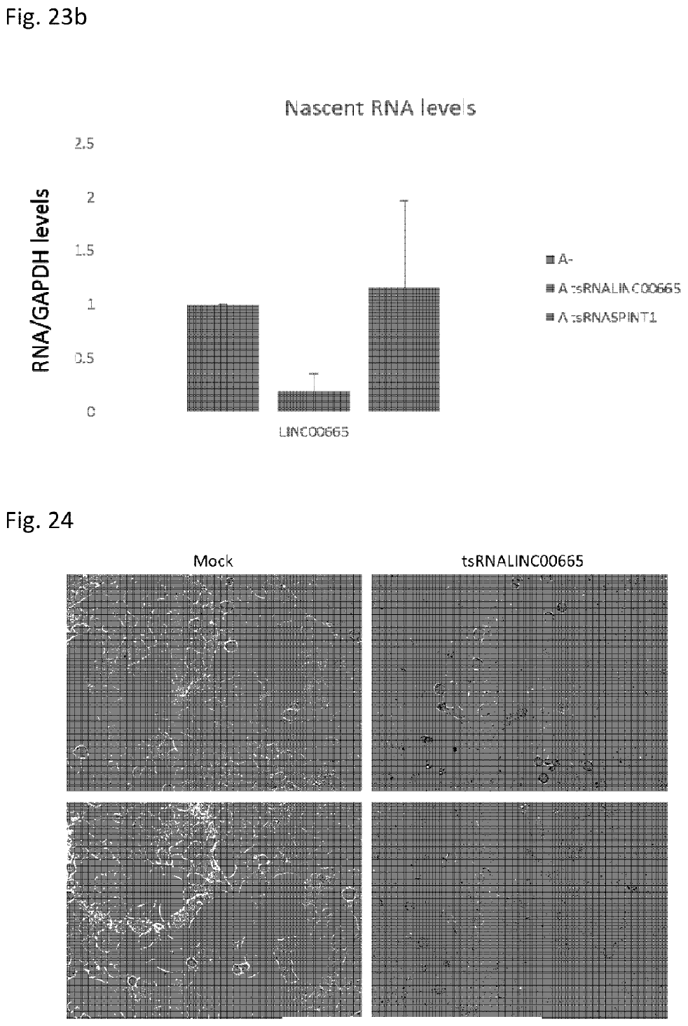

A549 cells were transfected with tsRNA LIN000665 for 24 hours. RNA was extracted for qRT-PCR. Primers targeting the exons were used for quantifying steady state RNA levels, while primers targeting the introns were used for quantifying nascent RNA levels. As is shown in FIGS. 23A and 23B, both steady state and nascent LINC0665 levels were reduced by transfection of tsRNA LINC0665 in A549 cells. As is shown in FIG. 24, A549 cells were transfected with tsRNA LIN000665 and imaged on day 3 with light microscopy. More dead cells were present in population of cells transfected with tsRNA LIN000665.

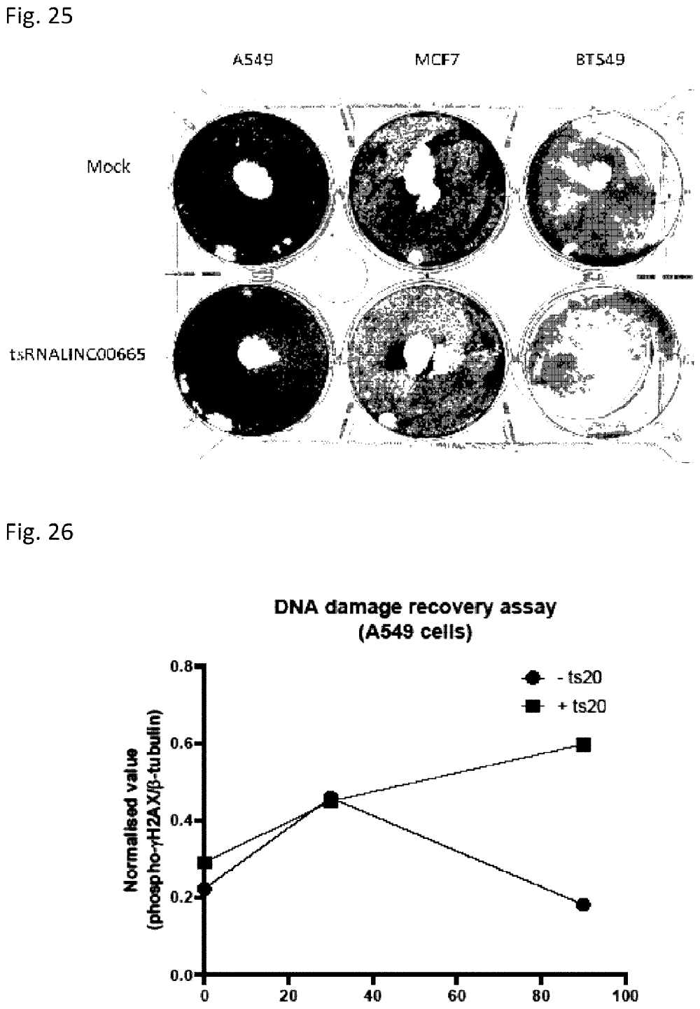

A549, MCR7 and BT549 cells were transfected with tsRNA LIN000665 and subjected to crystal violet staining on day 6. As is shown in FIG. 25, fewer live cells were present in populations of cells transfected with tsRNA LIN000665. A549 cells were transfected with tsRNA LIN000665 for 24 hours. Total protein was extracted at the 30-min and 90-min time points post-irradiation and subject to Western blotting. Signals of gamma-H2AX, used as a proxy for DNA damage, were quantified using a blot imager and normalised to beta-tubulin levels. As expected irradiated cells showed higher gamma-H2AX signals 30 mins post-irradiation; however, at the 90-min time point, cells transfected with ts20 (i.e. tsRNA targeting LIN000665) failed to repair DNA damage like the controls, suggesting that tsRNA LIN000665 is affecting genes involved in DNA damage response (shown in FIG. 26).

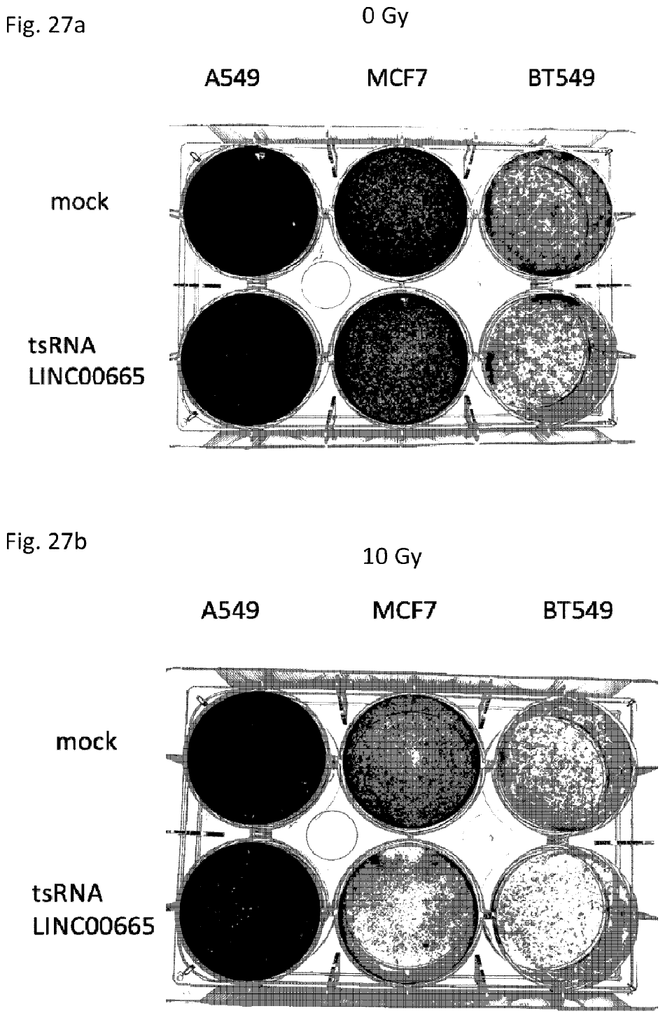

A549, MCF7 and BT549 were transfected with tsRNA LIN000665, irradiated at 10 Gy after 24 hours and finally subject to crystal violet staining at day 6. As is shown in FIGS. 27A and 27B, transfection of tsRNA LIN000665 into MCR7 and BT549 cells caused cell death; more cell death occurred when the cells were subjected to gamma-irradiation. However, this effect was magnified when transfection of tsRNA LIN000665 was combined with gamma-irradiation.

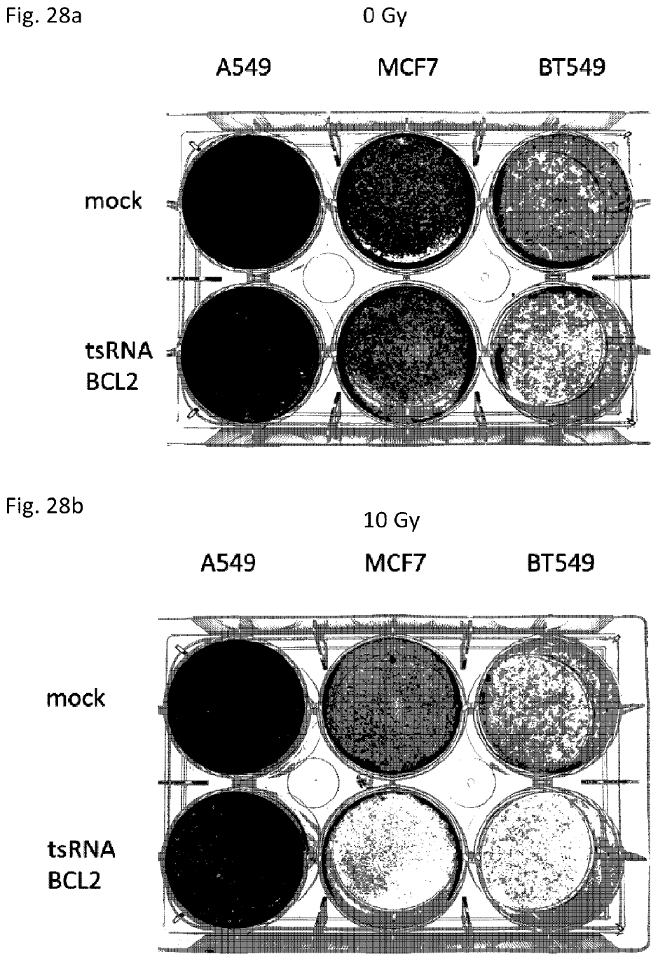

Finally, A549, MCF7 and BT549 were transfected with tsRNA BCL2, irradiated at 10 Gy after 24 hours and finally subject to crystal violet staining at day 6. As shown in FIGS. 28A and 28B, transfection of tsRNA BCL2 into MCR7 and BT549 cells caused cell death; more cell death occurred when the cells were subjected to gamma-irradiation. However, this effect was magnified when transfection of tsRNA LIN000665 was combined with gamma-irradiation.

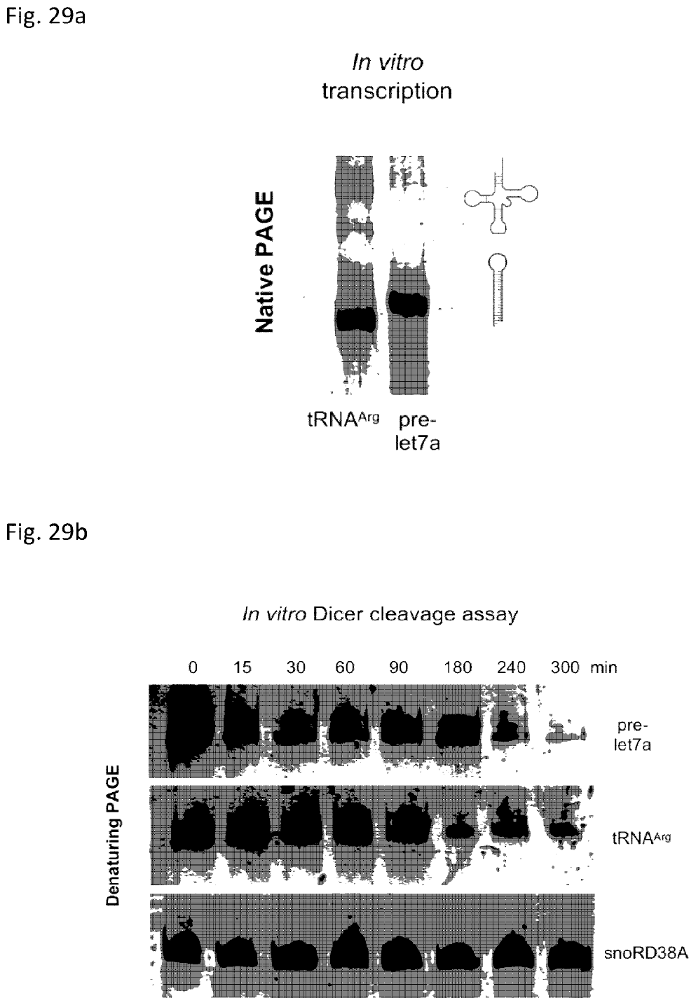

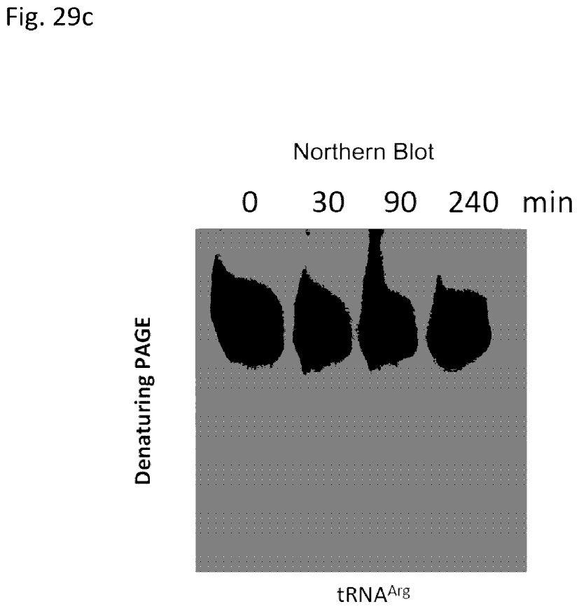

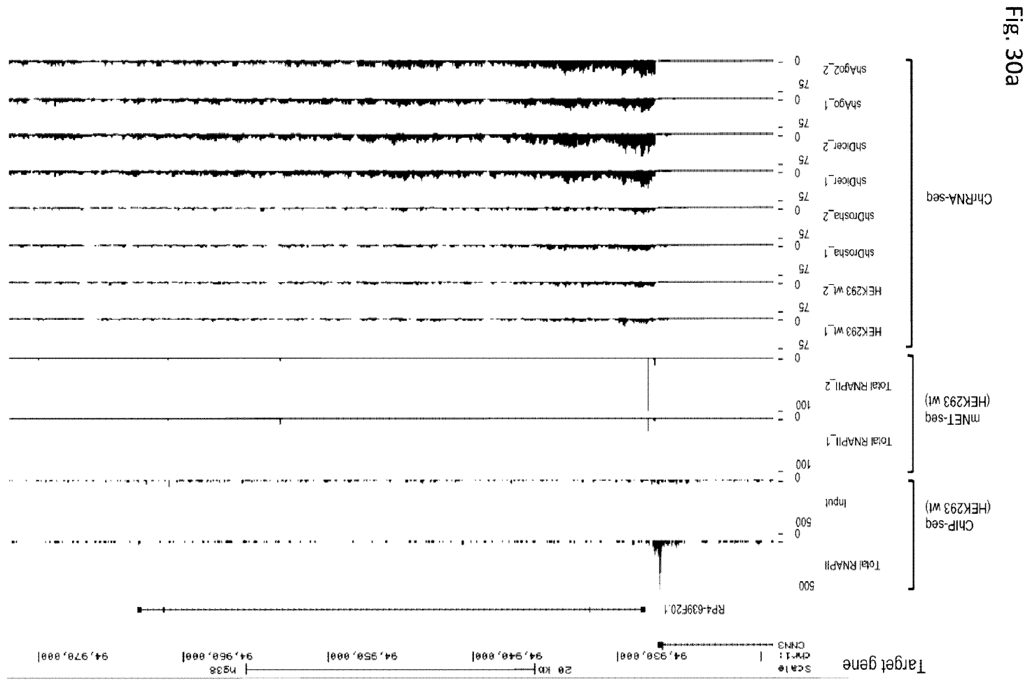

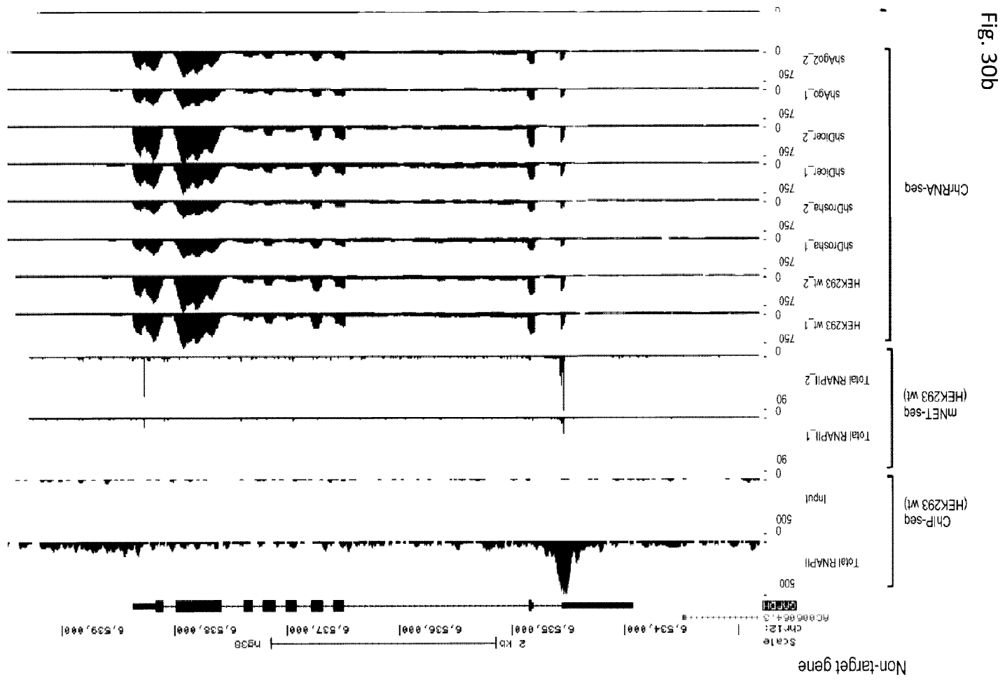

To verify the secondary structure prediction of this hairpin-like tRNA, we employed in vitro transcription of tRNA^{Arg-CCG-2-1} and the well-studied hairpin miRNA pre-let7a. As expected, transcription of pre-let7a resulted in a single band. tRNA^{Arg-CCG-2-1}, surprisingly, formed two bands: a minor one corresponding to the clover leaf structure and major one that ran close to the position of the pre-let7a control following native PAGE, suggesting that the structure is hairpin-like (FIG. 29a). It is important to note that the absence of RNA modifying enzymes in vitro may cause prominent alternative tRNA folding, whilst in vivo modified tRNAs may fold preferentially into clover leaf structures, hence the detection of a higher quantity of hair-pin like tRNAs in our experiment.

To test whether Dicer processes this hairpin-like tRNA into small RNAs, we in vitro transcribed tRNA^{Arg-CCG-2-1} pre-let7a and snoRD38A and incubated them with purified Dicer-TAP. We showed that the levels of pre-let7a substrate decreased in the presence of Dicer-TAP over time. Interestingly, hairpin-like tRNA^{Arg-CCG-2-1} substrate levels decreased in presence of Dicer, suggesting that Dicer may be processing hairpin-like tRNAs into small RNAs, whilst levels of snoRNA snoRD38A, used as negative control, did not change (FIG. 29b). However, we did not detect sRNA on the gel stained by SYBR gold, which might be a sensitivity issue. Subsequent northern analyses showed that the decreasing levels of tRNAs leads to production of sRNAs (FIG. 29c).

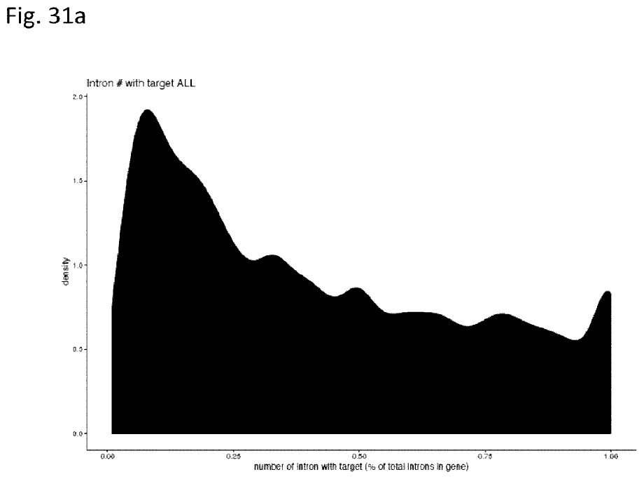

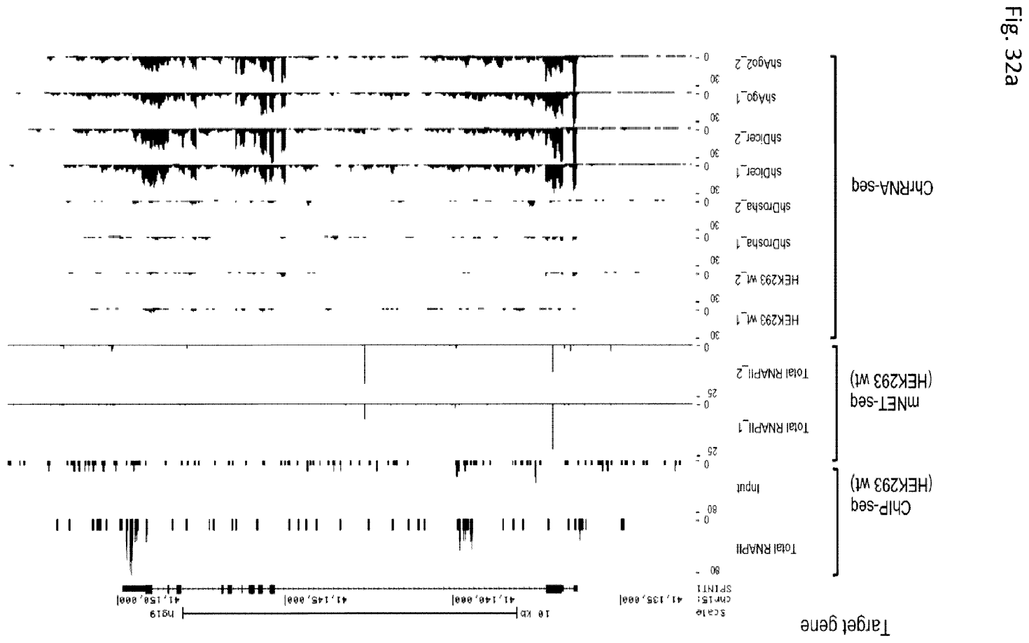

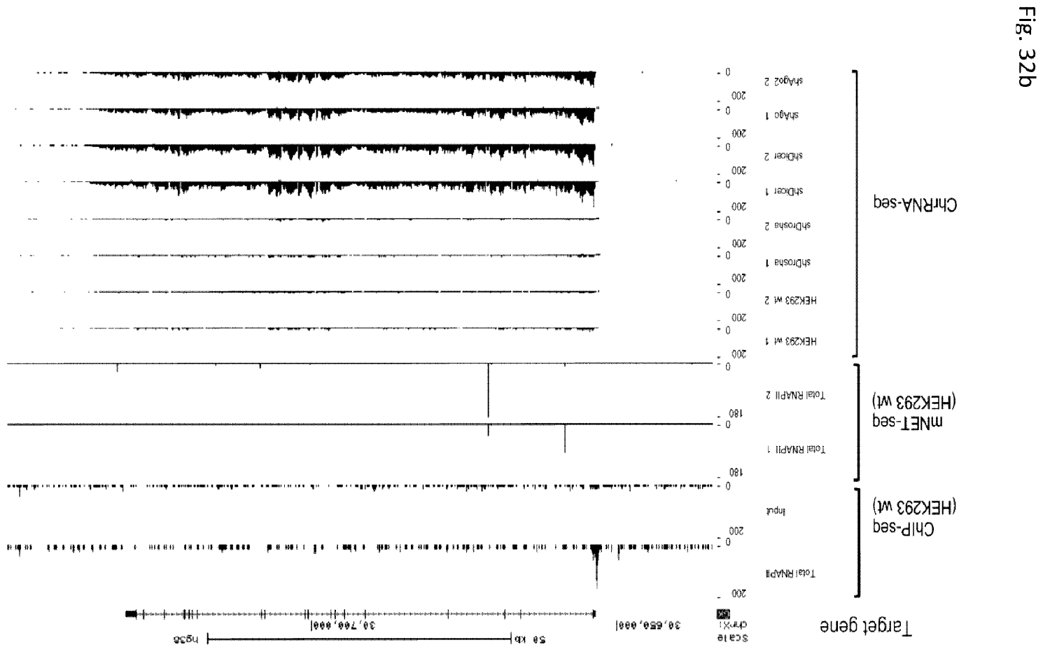

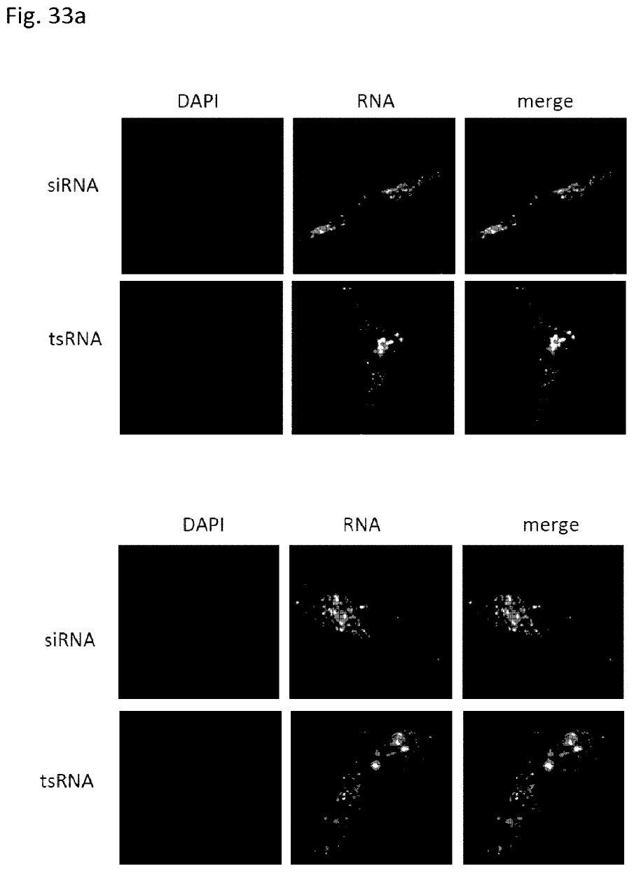

We subsequently performed miRanda (Enright et al., 2003) analysis on the chrRNA-seq data and show that tsRNAs target introns of protein coding genes as well as non-coding RNAs. Interestingly, there is a preference in targeting initial introns (FIG. 31a). Next, we investigated transcriptional state of the target genes in wt HEK293 cells. We aligned RNAP II ChIP-seq data (GSE126751), mammalian nascent elongating transcription (mNET-seq) data (Mayer et al., 2015) and chRNA-seq data (GSE126751). Close inspection of the snapshots of selected target genes RP4-639F20.1, SPINT1 and GK revealed the presence of RNAP II (ChIP-seq) as well as RNAP II protected nascent transcript signals (mNET-seq), but only very low levels of nascent RNA in wt cells. Nascent RNA levels of the target genes increased in Dicer and Ago2 knockdowns, but not in Drosha knockdown (FIGS. 30a, 32a and 32b). We also show combined snapshot for highly transcribed non-target gene GAPDH. We did observe any significant changes in GAPDH nascent RNA levels in wt, Drosha, Dicer and Ago2 knockdowns (FIG. 30b).

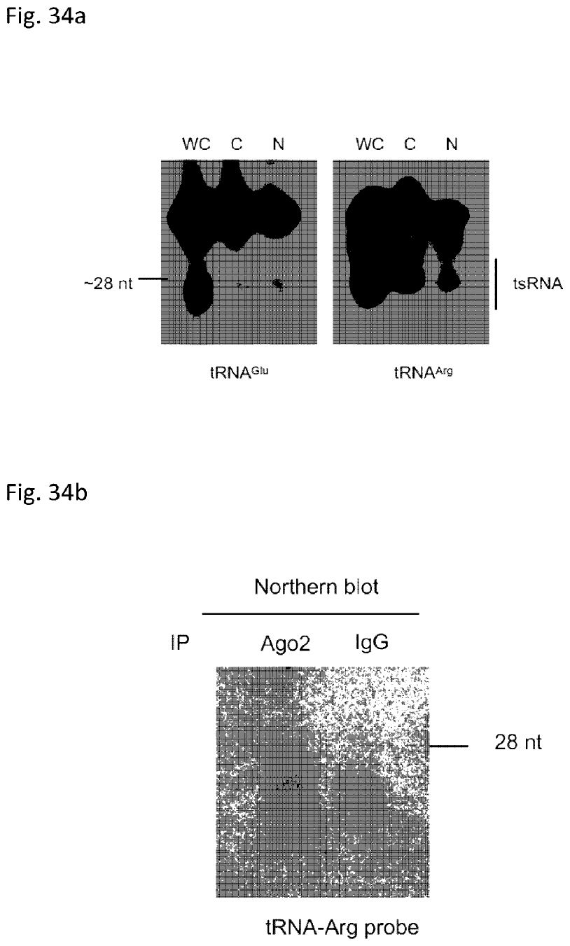

We hypothesised that if tsRNA target nascent RNA, they should be detectable in nucleus. In order to test whether tsRNA are localised in nucleus, we have in vitro fluorescently labelled synthetic tsRNA and siRNA targeting EGFR gene. Following transient transfection, we detected tsRNA in both nucleus and cytoplasm, whilst siRNA localised only in cytoplasm (FIG. 33a). Next, we performed fractionation, following Northern blot detecting two endogenous tsRNAs. Again we obtained signals from both fractions: nucleus and cytoplasm (FIG. 34a). To test whether Ago2 directly binds tsRNA, we performed immunoprecipitation of Ago2, followed by Northern blot detecting one of tsRNAs. Indeed we detected tsRNA signal in Ago2 pull down, but not in control IgG pull down (FIG. 34b).

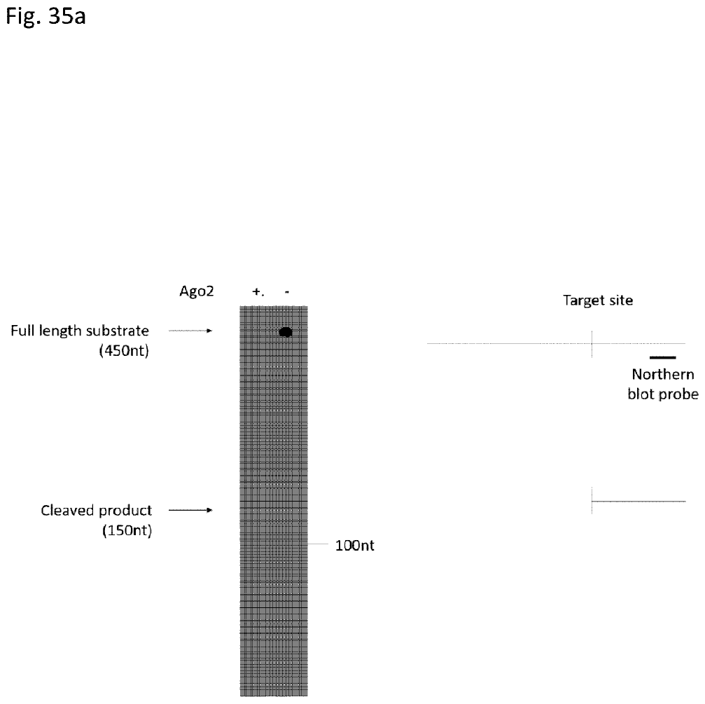

To assess the cleavage activity of Ago2 in this gene silencing pathway, we incubated FLAG-tagged AGO2 alongside a synthetic tsRNA with substrates bearing a sequence that is predicted to be targeted by the tsRNA. As shown by the northern blot, the full-length substrate was destabilised upon incubation with Ago2 and its targeting tsRNA, while the same full-length substrate remains intact in the absence of Ago2 (FIG. 35a).

These data demonstrate that tsRNAs target the intronic regions of specific genes to downregulate their expression through cleaving nascent RNA in an Ago2-dependent manner.

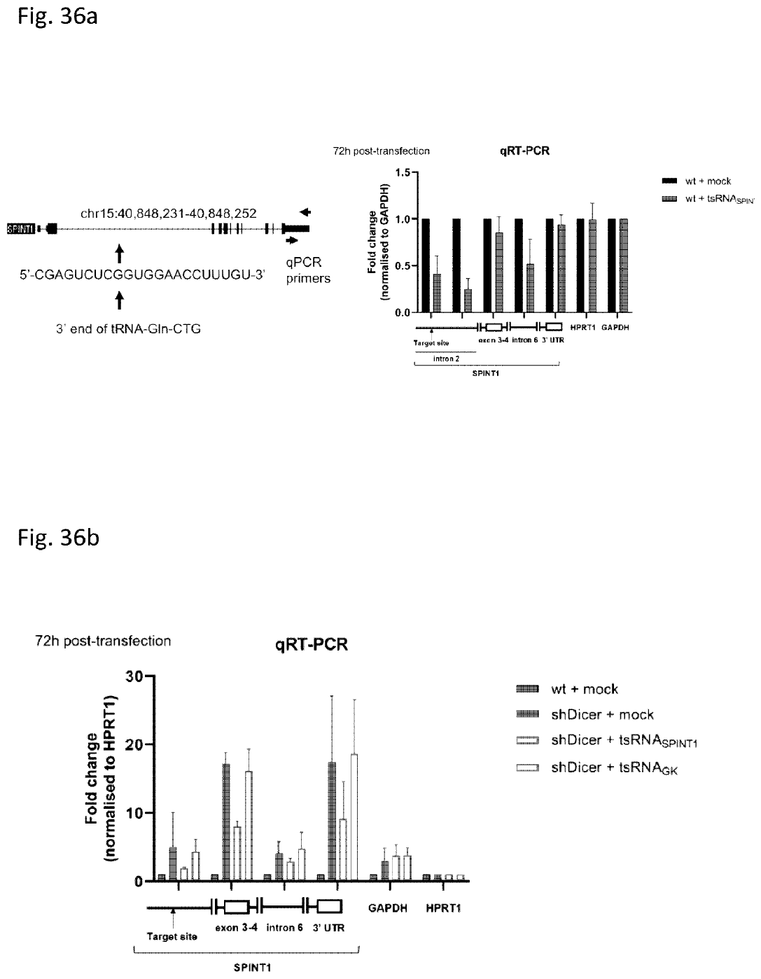

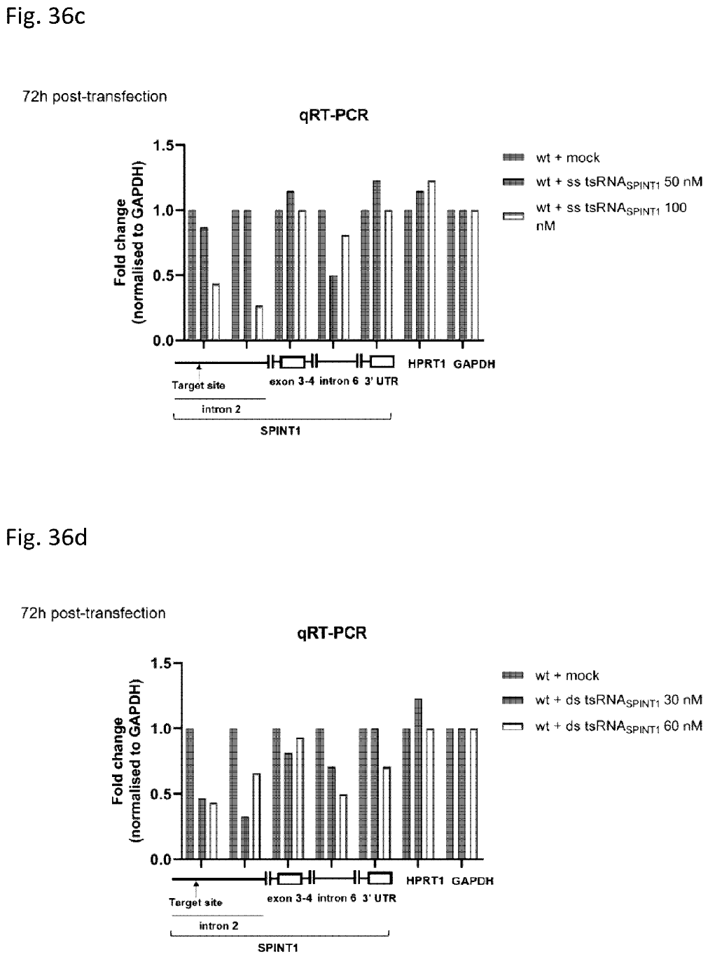

To confirm the intronic targeting further, we synthesized the tsRNA sequence predicted to target specifically the second intron of SPINT1. We transfected the synthetic tsRNAs into wt cells and observed a decrease in the nascent levels of SPINT1 pre-mRNA. The transfection of synthetic tsRNAs, however, did not lower the expression of the mature transcript (FIG. 36a). This may be because the levels of the mature transcript are very low under wt condition, detecting further silencing of such low levels can be difficult. The same synthetic tsRNA was then transfected into both wt and Dicer knockdown cells and the levels of SPINT1, nascent and mature, were assessed using qRT-PCR. We found that the upregulation of SPINT1 upon Dicer knockdown was significantly reduced following transfection of its intron-targeting tsRNA, (FIG. 36b). Additionally, we tested whether it is important that the synthetic tsRNAs are transfected in the single-stranded or double-stranded form. We found that both forms can lead to gene silencing of the targets, and in most cases, the effect is dose-dependent (FIGS. 36c and 36d).

It will be appreciated that numerous modifications to the above described method may be made without departing from the scope of the invention as defined in the appended claims.

### REFERENCES

1. Schmittgen, T. D. & Livak, K. J. Analyzing real-time PCR data by the comparative CT method. Nat. Protoc. 3, 1101-1108 (2008).
2. Burger, K. & Gullerova, M. Nuclear re-localization of Dicer in primary mouse embryonic fibroblast nuclei following DNA damage. PLOS Genet. 14, e1007151 (2018).
3. Nojima, T., Gomes, T., Carmo-Fonseca, M. & Proudfoot, N. J. Mammalian NET-seq analysis defines nascent RNA profiles and associated RNA processing genome-wide. Nat. Protoc. 11, 413-28 (2016).
4. White, E., Schlackow, M., Kamieniarz-Gdula, K., Proudfoot, N. J. & Gullerova, M. Human nuclear Dicer restricts the deleterious accumulation of endogenous double-stranded RNA. Nat Struct Mol Biol 21, 552-559 (2014).
5. Martin, M. Cutadapt removes adapter sequences from high-throughput sequencing reads. EMBnet.journal 17, 10 (2011).
6. Andrews, S. FastQC: a quality control tool for high throughput sequence data.
7. Oler, A. J. et al. Human RNA polymerase III transcriptomes and relationships to Pol II promoter chromatin and enhancer-binding factors. Nat. Struct. Mol. Biol. 17, 620-8
8. ENCODE Project Consortium. An integrated encyclopedia of DNA elements in the human genome. Nature 489, 57-74 (2012).
9. Langmead, B. & Salzberg, S. L. Fast gapped-read alignment with Bowtie 2. Nat. Methods 9, 357-9 (2012).
10. Li, H. et al. The Sequence Alignment/Map format and SAMtools. Bioinformatics 25, 2078-2079 (2009).
11. Quinlan, A. R. & Hall, I. M. BEDTools: a flexible suite of utilities for comparing genomic features. Bioinformatics 26, 841-2 (2010).
12. Zhang, Y. et al. Model-based Analysis of ChIP-Seq (MACS). Genome Biol. 9, R137 (2008).
13. Kent, W. J. et al. The human genome browser at UCSC. Genome Res. 12, 996-1006 (2002).
14. Langmead, B., Trapnell, C., Pop, M. & Salzberg, S. L. Ultrafast and memory-efficient alignment of short DNA sequences to the human genome. Genome Biol. 10, R25 (2009).
15. Hafner, M. et al. Transcriptome-wide Identification of RNA-Binding Protein and MicroRNA Target Sites by PAR-CLIP. Cell 141, 129-141 (2010).
16. Rybak-Wolf, A. et al. A variety of dicer substrates in human and C. elegans. Cell 159, 1153-1167 (2014).
17. Zerbino, D. R. et al. Ensembl 2018. Nucleic Acids Res. 46, D754-D761 (2018).
18. Bray, N. L., Pimentel, H., Melsted, P. & Pachter, L. Near-optimal probabilistic RNA-seq quantification. Nat. Biotechnol. 34, 525-527 (2016).
19. Love, M. I., Huber, W. & Anders, S. Moderated estimation of fold change and dispersion for RNA-seq data with DESeq2. Genome Biol. 15, 550 (2014).
20. Enright, A. J. et al. MicroRNA targets in Drosophila. Genome Biol. 5, R1 (2003).
21. Piñero, J. et al. DisGeNET: A comprehensive platform integrating information on human disease-associated genes and variants. Nucleic Acids Res. 45, D833-D839 (2017).
22. Smedley, D. et al. The BioMart community portal: an innovative alternative to large, centralized data repositories. Nucleic Acids Res. 43, W589-98 (2015).
23. R Core Team. R: A Language and Environment for Statistical Computing. (2016).
24. Kolde, R. pheatmap: Pretty Heatmaps.
25. Zuker, M. Mfold web server for nucleic acid folding and hybridization prediction. Nucleic Acids Res. 31, 3406-15 (2003).

1. An isolated tRNA-derived polynucleotide comprising a sequence that is complementary to an intronic region of a target gene or of a long non-coding RNA wherein said tRNA-derived polynucleotide is a tRNA-derived polynucleotide fragment that has 14 to 35 nucleotides (tsRNA).

2. The isolated tRNA-derived polynucleotide of claim 1 wherein the tsRNA is double stranded or single stranded.

3. The isolated tRNA-derived polynucleotide of claim 2 wherein the double stranded tsRNA is blunt ended.

4. The isolated tRNA-derived polynucleotide of claim 3 wherein the double stranded tsRNA comprises an overhang.

5. The isolated tRNA-derived polynucleotide of any preceding claim wherein the tRNA-derived polynucleotide is chemically modified.

6. The isolated tRNA-derived polynucleotide of claim 1 wherein the polynucleotide is tRNA.

7. The isolated tRNA-derived polynucleotide of claim 6 wherein the tRNA comprises a stem-loop/hairpin structure.

8. The isolated tRNA-derived polynucleotide according to any preceding claim wherein the polynucleotide binds an intronic region of the mRNA of the target gene thereby inhibiting gene expression.

9. The isolated tRNA-derived polynucleotide according to any preceding claim wherein the polynucleotide comprises a sequence that is at least 50, 60, 70, 80, 90 or 95% complementary to an intronic region of the target gene.

10. The isolated tRNA-derived polynucleotide according to any preceding claim wherein the target gene is associated with a pathological condition.

11. The isolated tRNA-derived polynucleotide according to claim 10 wherein the pathological condition is selected from cancer, autoimmune diseases, neurodegenerative diseases, metabolic diseases, respiratory diseases and cardiovascular diseases.

12. The isolated tRNA-derived polynucleotide according to any preceding claim wherein said tRNA and said tsRNA are located in the nucleus.

13. A vector comprising the isolated tRNA fragment according to any of claims 1 to 12.

14. A host cell comprising the isolated tRNA fragment according to any of claims 1 to 12 or the vector of claim 13.

15. A method of inhibiting expression of a target gene or of long non-coding RNA in a biological system, the method comprising:
introducing a tRNA-derived polynucleotide according to any of claims 1 to 12 or a vector according to claim 13 into the biological system.

16. The method according to claim 15 wherein the biological system is selected from a eukaryotic cell, such as a mammalian cell or a plant cell.

17. The method according to claim 15 or 16 wherein the method further comprises introducing an enzyme into the biological system which cleaves tRNA to produce tsRNA.

18. The method according to claim 17 wherein the enzyme is Dicer.

19. The method according to any of claims 15 to 18 preceding claim wherein the method further comprises introducing the tRNA-derived polynucleotide into the nucleus of a cell.

20. The method according to any of claims 15 to 19 wherein the method further comprises introducing an enzyme into the biological system which transports the tRNA-derived polynucleotide to the nucleus.

21. The method according to claim 20 wherein the enzyme comprises Argonaute 2 (Ago2).

22. The method according to any of claims 15 to 21 wherein the method is an in vitro or ex vivo method.

23. A pharmaceutical composition comprising a tRNA-derived polynucleotide according to any of claims 1 to 12 or a vector, e.g. according to claim 13 or a vector for modified cellular therapy that has been conditioned with tsRNA and a pharmaceutically acceptable carrier.

24. A tRNA-derived polynucleotide according to any of claims 1 to 12 or a pharmaceutical composition according to claim 23, for use as a medicament.

25. A tRNA-derived polynucleotide according to any of claims 1 to 12 or a pharmaceutical composition according to claim 23, for use in treating a disease which can be ameliorated by inhibiting expression of the target gene.

26. The tRNA-derived polynucleotide or a pharmaceutical composition for use according to claim 24 or 25 wherein the disease is selected from cancer, autoimmune diseases, neurodegenerative diseases, metabolic diseases, respiratory diseases and cardiovascular diseases.

27. The tRNA-derived polynucleotide or a pharmaceutical composition for use according to claim 27 wherein the disease is selected from cancer and the tRNA-derived polynucleotide is administered together with a second therapy, such as an anti-cancer therapy.

28. A method for the treatment of cancer, autoimmune diseases, neurodegenerative diseases, metabolic diseases, respiratory diseases and cardiovascular diseases comprising administering an effective amount of tRNA-derived polynucleotide according to any of claims 1 to 12 or a pharmaceutical composition according to claim 23 to a subject in need thereof.

29. Use of a tRNA-derived polynucleotide according to any of claims 1 to 12 for inhibiting expression of a gene or of a long non-coding RNA in a biological system.

30. The use according to claim 29, where the use is performed in vitro or ex vivo.

31. A kit comprising a tRNA-derived polynucleotide according to any of claims 1 to 12 or a pharmaceutical composition according to claim 23.

32. A method for identifying a tsRNA fragment that mediates RNA interference of a target gene said method comprising
a) providing a sample;
b) isolating a tsRNA fragment having between around 14 and 35 nucleotides from said sample;
c) characterising the tsRNA fragment to determine sequence identity or similarity with the target gene and;
d) identifying a tsRNA fragment that comprises a sequence that is complementary to an intronic region of a target gene.

33. A tsRNA fragment that mediates RNA interference obtained or obtainable by the method of claim 32.

34. A method for producing tsRNA fragment that mediates RNA interference comprising identifying a tsRNA fragment according to claim 32.

35. A combination therapy comprising administration of a tsRNA-derived polynucleotide according to any of claims 1 to 12 and another therapy, such as an anti-cancer therapy.

36. The combination therapy of claim 35 wherein the anticancer therapy is radiotherapy or chemotherapy.

37. A method of mediating target specific RNA interference, the method comprising:
introducing a tRNA-derived polynucleotide according to any of claims 1 to 12 into a biological system.

38. A method of detecting a disease, the method comprising;
a) Detecting the presence of a tRNA-derived tsRNA fragment that has 14 to 35 nucleotides and is complementary to an intronic region of a target gene or of a long non-coding RNA in a sample;
b) Quantifying the amount of the tsRNA present in the sample;
c) Comparing the amount of tsRNA present in the sample to a reference value and;
d) Assessing the presence of absence of the disease.

39. The method according to claim 38, wherein the reference is the amount of tsRNA in healthy cells or diseased cells.

40. The method according to claim 38 or 39 wherein the disorder is selected from cancer, autoimmune diseases, neurodegenerative diseases, metabolic diseases, respiratory diseases and cardiovascular diseases.

41. The method according to any of claims 38 to 40, wherein the isolated tsRNA is quantified by RT-PCR.

42. The method according to any of claims 38 to 40, wherein the sample is a blood sample, tissue sample, exosomes, urine, saliva or CSF.

43. The method according to claim, wherein the method is performed in vitro or ex vivo.

44. A computer implemented method for generating a candidate tRNA-derived polynucleotide that comprises a sequence that is complementary to an intronic region of the target gene, according to claim 1 or 2, and is capable of inhibiting gene expression of the target gene said method comprising:
a) Determining the inherent features of the tRNA from which the said polynucleotide is derived;
b) Determining the inherent features of the binding sites within an intronic region of a target gene, to which the tRNA-derived polynucleotide binds;
c) Generating a dataset comprising known tRNA-derived polynucleotide and binding sites;
d) Using the dataset to define a training dataset to identify any patterns in structure, nucleotide content, position within an intron, primary, secondary or tertiary structure or gene targets;
e) Screening a genome sequence using the training dataset to identify candidate binding sites within intronic region of the target gene and;
f) Using the output generating a candidate tRNA-derived polynucleotide that comprises a sequence that is complementary to an intronic region of the target gene.

45. The method according to claim 44, wherein the inherent features include sequence, secondary structure and/or location within the genome.

46. A computer system for identifying one or more unique tRNA-derived polynucleotide sequences in a genome of a eukaryotic organism, the system comprising:
I. a memory unit configured to receive and/or store sequence information of the genome; and
II. one or more processors alone or in combination programmed to perform a method according to claim 44 or 45.
